SEQUENCE LISTING (23 sequences)

SEQ ID NO: 1 - DNA, 1940 bp, Arabidopsis thaliana
gattttctttttggtttgatgcagtgaggagaggaaatgtcggatcttcagacaccgcttgtgaggcccaagaggaagaagacttgggttgattactttgtcaagttcagatggatcattgtcatcttcatcgtccttccattctcagccacattctacttcctcatctacctcggggacatgtggtcagagtccaagtcctttgagaaacgtcagaaggaacacgacgagaatgtcaagaaagtcatcaaaaggcttaagggtagggatgcttccaaggacgggcttgtctgcactgctcgtaagccctggatcgctgttggaatgaggaacgttgactacaagagagcccggcatttcgaggttgacttgggggagttccgtaacatccttgagatcaacaaggagaagatgactgctagagtggagcctcttgttaacatgggacagatttcccgtgctaccgtcccaatgaacctgtctctcgctgttgttgctgagcttgatgaccttaccgttggtggacttatcaatggatatggtattgaaggaagctctcacatctacggtttgtttgctgataccgttgaggcttacgagattgttcttgcgggtggagagcttgtccgcgccacaagggataatgagtattctgatctttactacgcaatcccgtggtcgcaaggaactcttggactccttgtagctgctgagatcaggcttattaaagtcaaggagtacatgagactcacttacataccagtcaagggtgatcttcaagccttagctcaaggttacattgattcttttgctcccaaagacggtgacaagtcgaaaatcccggatttcgtcgaaggcatggtttacaatccaacggaaggagtgatgatggttggaacatatgcatctaaagaagaggcaaagaagaaagggaacaaaatcaacaatgtgggatggtggttcaagccgtggttctaccagcacgcgcagaccgccctgaaaaagggacagtttgttgagtacatcccaactcgtgaatactaccacaggcacacaaggtgcttgtactgggaagggaagcttattcttccatttggtgatcagttctggtttaggtacctcttaggttggttgatgcctccaaaggtctctcttcttaaggccactcaaggtgaagctatcaggaactattaccatgatatgcatgttattcaggatatgcttgttcctctttacaaggttggcgatgcactcgaatgggtccaccgcgaaatggaggtgtatccaatttggctttgcccacacaaactcttcaagcagccaatcaaaggccaaatctacccagagccaggcttcgagtacgaaaacagacaaggagacacagaagatgcacagatgtacactgatgttggagtctactacgcacctggctgtgtcctaagaggtgaagagtttgatggatcagaagcagtgcgtaggatggagaaatggctgatagagaaccatggattccagcctcagtacgcggtgtctgagctcgacgagaagagcttctggagaatgtttaatggtgaattgtatgaggagtgccgcaagaagtatagagctattggaacgttcatgagtgtttactacaagtccaagaaaggaaggaagactgagaaagaagttagagaagccgaacaagctcatctcgaaactgcttatgccgaggcagattaagtaataataagccttggtttgatgtaatggctcgggatgattattgttattcctagtagtgtctgttatgttgttgtttcctaatttcagttttagtgtcttgaagactcttgttgttgttgtttgcttcttgttgttgtttgactcattctggtttgagtggattttagagtttaaaacttttggatttgcctatgaaattgaaggtttccggtttt

SEQ ID NO: 2 - protein, 561 aa, Arabidopsis thaliana
MSDLQTPLVRPKRKKTWVDYFVKFRWIIVIFIVLPFSATFYFLIYLGDMWSESKSFEKRQKEHDENVKKVIKRLKGRDASKDGLVCTARKPWIAVGMRNVDYKRARHFEVDLGEFRNILEINKEKMTARVEPLVNMGQISRATVPMNLSLAVVAELDDLTVGGLINGYGIEGSSHIYGLFADTVEAYEIVLAGGELVRATRDNEYSDLYYAIPWSQGTLGLLVAAEIRLIKVKEYMRLTYIPVKGDLQALAQGYIDSFAPKDGDKSKIPDFVEGMVYNPTEGVMMVGTYASKEEAKKKGNKINNVGWWFKPWFYQHAQTALKKGQFVEYIPTREYYHRHTRCLYWEGKLILPFGDQFWFRYLLGWLMPPKVSLLKATQGEAIRNYYHDMHVIQDMLVPLYKVGDALEWVHREMEVYPIWLCPHKLFKQPIKGQIYPEPGFEYENRQGDTEDAQMYTDVGVYYAPGCVLRGEEFDGSEAVRRMEKWLIENHGFQPQYAVSELDEKSFWRMFNGELYEECRKKYRAIGTFMSVYYKSKKGRKTEKEVREAEQAHLETAYAEAD

SEQ ID NO: 3 - DNA, 1093 bp, Arabidopsis thaliana
ttccattatttgtgaatttatttacagtaaacaatcagacccaaaaaataaaaaattccaattaaagttctccaattggagttgattctgcccttattagagagatctgatctttccccaattccataacccgaaaaatggaagaaatcgccgataaaaccttcttccgatactgtctcctcactcttattttcgccggcccaccaaccgccgtccttctgaaattcctccaagctccttacggtaaacacaaccgtaccggatggggtcccaccgtatctccaccgattgcttggttcgtcatggagagcccaaccttgtggctcactctcctcctcttcccctttggtcgtcacgctctcaaccctaaatctctacttctattctctccttatctcattcattacttccaccgcaccatcatttaccctcttcgcctcttccgcagctccttccccgccggtaaaaacggatttccgatcaccatcgccgccttggctttcacctttaatctcctcaatggttatatccaggcgaggtgggtttcgcattacaaggatgactacgaagacggaaactggttctggtggcggtttgttatcggtatggtggttttcataaccggcatgtatataaatatcacgtcggaccggactttggtacgattgaagaaagagaaccggggaggttatgtgataccgagaggaggctggttcgagttggtaagctgtccgaattattttggagaggcgattgagtggttgggctgggctgttatgacttggtcttgggccggtattggattttttctgtacacgtgttccaatttgtttccgcgtgcacgtgcgagtcacaagtggtacattgccaagttcaaggaagagtatcccaagactcgtaaagctgttattccttttgtgtactgagaattgagaaagttgaaaactagtttatcatatgttatgtgtcaatttgtttccaaactacctttgtcaaaatttccagtaaccggtttaattccaacacggtttagatcttatgttggtatcttcaacaatgcacaacaaactgtgtattctttagacaaatttta

SEQ ID NO: 4 - protein, 262 aa, Arabidopsis thaliana
MEEIADKTFFRYCLLTLIFAGPPTAVLLKFLQAPYGKHNRTGWGPTVSPPIAWFVMESPTLWLTLLLFPFGRHALNPKSLLLFSPYLIHYFHRTIIYPLRLFRSSFPAGKNGFPITIAALAFTFNLLNGYIQARWVSHYKDDYEDGNWFWWRFVIGMVVFITGMYINITSDRTLVRLKKENRGGYVIPRGGWFELVSCPNYFGEAIEWLGWAVMTWSWAGIGFFLYTCSNLFPRARASHKWYIAKFKEEYPKTRKAVIPFVY

SEQ ID NO: 5 - DNA, 1740 bp, Arabidopsis thaliana
gaagtccgattcccaatcttaaagacaaagccattagaaagagaaagtgagtgagagagagagagaaactagctccatgttcgaaacagagcatcatactctcttacctcttcttcttctcccatcgcttttgtctcttcttctcttcttgattctcttgaagagaagaaatagaaaaaccagattcaatctacctccgggtaaatccggttggccatttcttggtgaaaccatcggttatcttaaaccgtacaccgccacaacactcggtgacttcatgcaacaacatgtctccaagtatggtaagatatatagatcgaacttgtttggagaaccaacgatcgtatcagctgatgctggacttaatagattcatattacaaaacgaaggaaggctctttgaatgtagttatcctagaagtataggtgggattcttgggaaatggtcgatgcttgttcttgttggtgacatgcatagagatatgagaagtatctcgcttaacttcttaagtcacgcacgtcttagaactattctacttaaagatgttgagagacatactttgtttgttcttgattcttggcaacaaaactctattttctctgctcaagacgaggccaaaaagtttacgtttaatctaatggcgaagcatataatgagtatggatcctggagaagaagaaacagagcaattaaagaaagagtatgtaactttcatgaaaggagttgtctctgctcctctaaatctaccaggaactgcttatcataaagctcttcagtcacgagcaacgatattgaagttcattgagaggaaaatggaagagagaaaattggatatcaaggaagaagatcaagaagaagaagaagtgaaaacagaggatgaagcagagatgagtaagagtgatcatgttaggaaacaaagaacagacgatgatcttttgggatgggttttgaaacattcgaatttatcgacggagcaaattctcgatctcattcttagtttgttatttgccggacatgagacttcttctgtagccattgctctcgctatcttcttcttgcaagcttgccctaaagccgttgaagagcttagggaagagcatcttgagatcgcgagggccaagaaggaactaggagagtcagaattaaattgggatgattacaagaaaatggactttactcaatgtgttataaatgaaactcttcgattgggaaatgtagttaggtttttgcatcgcaaagcactcaaagatgttcggtacaaaggatacgatatccctagtgggtggaaagtgttaccggtgatctcagccgtacatttggataattctcgttatgaccaacctaatctctttaatccttggagatggcaacagcaaaacaacggagcgtcatcctcaggaagtggtagtttttcgacgtggggaaacaactacatgccgtttggaggagggccaaggctatgtgctggttcagagctagccaagttagaaatggcagtgtttattcatcatctagttcttaaattcaattgggaattagcagaagatgataaaccatttgcttttccttttgttgattttcctaacggtttgcctattagggtttctcgtattctgtaaaaaaaaaaaaagatgaaagtatttttattctcttcttttttttttgataattttaaatcattttttttgcccaatgatatataaaaatttggataaataatattattggatattcgtttttt

SEQ ID NO: 6 - protein, 513 aa, Arabidopsis thaliana
MFETEHHTLLPLLLLPSLLSLLLFLILLKRRNRKTRFNLPPGKSGWPFLGETIGYLKPYTATTLGDFMQQHVSKYGKIYRSNLFGEPTIVSADAGLNRFILQNEGRLFECSYPRSIGGILGKWSMLVLVGDMHRDMRSISLNFLSHARLRTILLKDVERHTLFVLDSWQQNSIFSAQDEAKKFTFNLMAKHIMSMDPGEEETEQLKKEYVTFMKGVVSAPLNLPGTAYHKALQSRATILKFIERKMEERKLDIKEEDQEEEEVKTEDEAEMSKSDHVRKQRTDDDLLGWVLKHSNLSTEQILDLILSLLFAGHETSSVAIALAIFFLQACPKAVEELREEHLEIARAKKELGESELNWDDYKKMDFTQCVINETLRLGNVVRFLHRKALKDVRYKGYDIPSGWKVLPVISAVHLDNSRYDQPNLFNPWRWQQQNNGASSSGSGSFSTWGNNYMPFGGGPRLCAGSELAKLEMAVFIHHLVLKFNWELAEDDKPFAFPFVDFPNGLPIRVSRIL

SEQ ID NO: 7 - DNA, 1713 bp, Arabidopsis thaliana
cccactctccccttctccattaatactctctctccctcatcctctcttcttctctcatcatcatcttcttcttcaatggccttcaccgcttttctcctcctcctctcttccatcgccgccggcttcctcctcctactccgccgtacacgttaccgtcggatgggtctgcctccgggaagccttggtctccctctgataggagagacttttcagctgatcggagcttacaaaacagagaaccctgagcctttcatcgacgagagagtagcccggtacggttcggttttcatgacgcatctttttggtgaaccgacgattttctcagctgacccggaaacgaaccggtttgttcttcagaacgaagggaagctttttgagtgttcttatcctgcttccatttgtaaccttttggggaaacactctctgcttcttatgaaaggttctttgcataaacgtatgcactctctcaccatgagctttgctaattcttcaatcattaaagaccatctcatgcttgatattgaccggttagtccggtttaatcttgattcttggtcttctcgtgttctcctcatggaagaagccaaaaagataacgtttgagctaacggtgaagcagttgatgagctttgatccaggggaatggagtgagagtttaaggaaagagtatcttcttgtcatcgaaggcttcttctctcttcctctccctctcttctccaccacttaccgcaaagccatccaagcgcggaggaaggtggcggaggcgttgacggtggtggtgatgaaaaggagggaggaggaggaagaaggagcggagagaaagaaagatatgcttgcggcgttgcttgcggcggatgatggattttccgatgaagagattgttgacttcttggtggctttacttgtcgccggttatgaaacaacctccacgatcatgactctcgccgtcaaatttctcaccgagactcctttagctcttgctcaactcaaggaagagcatgaaaagattagggcaatgaagagtgattcgtatagtcttgaatggagtgattacaagtcaatgccattcacacaatgtgtggttaatgagacgctacgagtggctaacatcatcggcggtgttttcagacgtgcaatgacggatgttgagatcaaaggttataaaattccaaaagggtggaaagtattctcatcgtttagagcggttcatttagacccaaaccacttcaaagatgctcgcactttcaacccttggagatggcagagcaactcggtaacgacaggcccttctaatgtgttcacaccgtttggtggagggccaaggctatgtcccggttacgagctggctagggttgcactctctgttttccttcaccgcctagtgacaggcttcagttgggttcctgcagagcaagacaagctggttttctttccaactacaagaacgcagaaacggtacccgatcttcgtgaagcgccgtgattttgctacttgaagaagaagagacccatctgattttatttatagaacaacagtatttttcaggattaatttcttcttctttttttgcctccttgtgggtctagtgtttgacaataaaagttatcattactctataaagccttagcttctgtgtacataaaaaaaaaaaacttttgtttaccttatgcttgcataaatctcttctgcttcaatggttcaaacatgagttgct

SEQ ID NO: 8 - protein, 472 aa, Arabidopsis thaliana
MAFTAFLLLLSSIAAGFLLLLRRTRYRRMGLPPGSLGLPLIGETFQLIGAYKTENPEPFIDERVARYGSVFMTHLFGEPTIFSADPETNRFVLQNEGKLFECSYPASICNLLGKHSLLLMKGSLHKRMHSLTMSFANSSIIKDHLMLDIDRLVRFNLDSWSSRVLLMEEAKKITFELTVKQLMSFDPGEWSESLRKEYLLVIEGFFSLPLPLFSTTYRKAIQARRKVAEALTVVVMKRREEEEEGAERKKDMLAALLAADDGFSDEEIVDFLVALLVAGYETTSTIMTLAVKFLTETPLALAQLKEEHEKIRAMKSDSYSLEWSDYKSMPFTQCVVNETLRVANIIGGVFRRAMTDVEIKGYKIPKGWKVFSSFRAVHLDPNHFKDARTFNPWRWQSNSVTTGPSNVFTPFGGGPRLCPGYELARVALSVFLHRLVTGFSWVPAEQDKLVFFPTTRTQKRYPIFVKRRDFAT

SEQ ID NO: 9 - DNA, 2135 bp, Arabidopsis thaliana
attgagaaaacccttttaaaattctctatcgatctcatcacaaattttgctatatccacaattcatgtgcttaggcatatagttattcccaagaaaccggtttaactgtttacgtatgcaacctccggcaagcgcaggacttttccggtcgccggaaaatctcccttggccttataattacatggattatttggtcgctggtttcttggttttgacggccggaatacttctccgtccatggctctggttacgtctacgaaactcgaaaacgaaagatggagatgaagaagaagataatgaggagaagaagaagggaatgattccaaacggaagcttaggctggccggtgatcggagaaaccctaaacttcatcgcttgtggttattcttctcggcctgttaccttcatggacaaacgaaagtctttatacgggaaagtgttcaaaacgaacataatagggacaccaatcataatatcaaccgatgcagaggtgaataaagtggtgctccaaaaccatgggaacacatttgtccctgcataccctaaatcaattacggaactacttggagaaaactctattctcagcatcaatggacctcatcaaaaaaggcttcacacgctcattggcgcgttcctcagatctcctcacctcaaagaccggatcactcgagacattgaggcctcggttgttctcactttggcgtcttgggctcaacttccattggttcatgttcaggatgagatcaaaaagatgacgtttgagatattagtaaaagtgttgatgagcacatctcctggtgaagatatgaacattctcaaacttgagttcgaagaattcatcaaaggtttgatttgtatcccaatcaaattccctggcactagactctacaaatccttaaaggcgaaagagaggttaataaagatggtaaaaaaggttgtggaggagagacaagtggcgatgacaacgacgtctccggcaaatgacgtggtggacgtacttctaagagacggtggtgattcagagaagcaatctcaaccgtcagatttcgtcagcggaaagatcgtagagatgatgatacccggagaggaaacaatgccaacggcgatgaccttggctgtcaaattcttaagtgacaaccccgtcgctctagccaaactcgtggaggagaatatggagatgaagaggcgtaaattggaattgggagaagaatacaagtggaccgattatatgtctctctcttttactcaaaatgtgataaacgaaacgcttagaatggctaacattattaacggggtgtggaggaaagctctcaaggatgtagaaattaaaggttacttaataccgaaaggatggtgtgtattggcatcattcatatcggttcacatggatgaagacatttatgataatccctatcaattcgatccgtggagatgggacagaattaatggatcggcaaacagcagtatttgcttcacaccctttggtggtgggcaaaggctatgtcctggtttagagctgtcgaagctcgaaatatccatctttcttcaccaccttgtaacccggtacagttggacggctgaggaagacgagatagtgtcatttccgactgtgaagatgaagcggaggctcccgatccgagtggctactgtagatgatagtgcttctccgatctcacttgaagatcattaatagatcatttcaaagaacaaaactgtttgtgcaaagaggaagcagagaagtaaacaaatgatcttattaacaaatagtagagaagagaagcaaacaagattggtgggtaagacagaaagagccatacgtacagctagtgatggctcaaagatgagagattctaattataattttttttgtttgtcatgtcaaattataagcgttggttaggttgtccctttctcttttatttatcgtaccaaacgcaagttgagatatgattccatatatatggatgatagatatgtatattaatatataccttcttctttttttggcagctattaacattatgcctatctcgtctctatctagcttttgttttatgctcgagaaaatttagacgaaaatatatcgaaaaatcgacgactagtctcgatcgataatatgatgatcacttcattta

SEQ ID NO: 10 - protein, 524 aa, Arabidopsis thaliana
MQPPASAGLFRSPENLPWPYNYMDYLVAGFLVLTAGILLRPWLWLRLRNSKTKDGDEEEDNEEKKKGMIPNGSLGWPVIGETLNFIACGYSSRPVTFMDKRKSLYGKVFKTNIIGTPIIISTDAEVNKVVLQNHGNTFVPAYPKSITELLGENSILSINGPHQKRLHTLIGAFLRSPHLKDRITRDIEASVVLTLASWAQLPLVHVQDEIKKMTFEILVKVLMSTSPGEDMNILKLEFEEFIKGLICIPIKFPGTRLYKSLKAKERLIKMVKKVVEERQVAMTTTSPANDVVDVLLRDGGDSEKQSQPSDFVSGKIVEMMIPGEETMPTAMTLAVKFLSDNPVALAKLVEENMEMKRRKLELGEEYKWTDYMSLSFTQNVINETLRMANIINGVWRKALKDVEIKGYLIPKGWCVLASFISVHMDEDIYDNPYQFDPWRWDRINGSANSSICFTPFGGGQRLCPGLELSKLEISIFLHHLVTRYSWTAEEDEIVSFPTVKMKRRLPIRVATVDDSASPISLEDH

SEQ ID NO: 11 - DNA, 1914 bp, Arabidopsis thaliana
gaaagaaagtcccaaaaacaaaaaaccaattttaaaaaacacaaacccattcgtgcgtctctggctaattaagttaaaacactaatggacacttcttcttcacttttgttcttctccttcttcttctttatcatcatcgtcatcttcaacaagatcaacggtctcagatcatccccagcttcaaagaaaaaacttaatgatcatcatgttacatcccagagtcacggaccaaagtttccacacggaagcttgggatggcccgtcatcggtgaaaccatcgagttcgtctcttctgcttactcagaccgtcctgagagtttcatggacaagcgtcgtctcatgtatgggagagtgtttaagtcgcatatttttggaacggcgacgatcgtgtcgacggatgctgaagtgaacagagccgttttacagagcgactcgacagctttcgtgccgttttacccaaaaacggtaagggagctaatgggaaaatcgtcgatacttcttatcaacgggagtttacatagacggttccatggattagtcggttctttcttaaagtcgccacttctcaaagctcaaatcgttagagacatgcacaagtttttgtcggaatccatggatctatggtccgaggaccaacctgtgctcctccaagacgtctccaagactgttgcattcaaagtacttgccaaggcattgataagtgtagagaaaggagaagatttagaagagctaaagagagagtttgaaaatttcatatcaggactcatgtcattaccaattaacttccctggaacgcaactccatagatctctccaagctaagaagaatatggtgaagcaagttgaaagaatcatagaaggcaaaattaggaaaacaaagaacaaggaggaagatgatgttattgcaaaggatgttgtggatgtgttgcttaaggactcaagtgaacatttaactcacaatttgattgctaacaatatgatcgacatgatgatccctggccacgattctgtccctgtcctcattacccttgccgtcaaattcctctctgattctcctgctgccctcaatctcctaacggaagaaaacatgaagctgaaaagtttgaaggaattgacaggagagccactatattggaatgactacttgtcgttaccttttacacaaaaggtgattacagagacactgagaatgggaaatgttataattggagtgatgagaaaggcgatgaaagatgttgaaataaaaggatatgtgataccaaaaggatggtgtttcttggcctatctcagatcagttcatcttgataagctttattatgagtctccctacaaatttaatccctggagatggcaagaaagggacatgaacacgagtagtttcagtccttttggaggtggtcagagattgtgccctggtctcgatttggctcgtcttgaaacttcagtttttcttcaccatcttgtcactcgcttcagatggatagcagaagaagacacaatcataaacttcccaacggtgcatatgaagaacaaattacccatttggatcaaaagaatataaatcatttttcttaagtgaaaaatggagaatcagttttgcattatcaaactctaaaaggttaaagaagtcacacgagtgttttgtaagtgggaagtgaaaaagatagtgagacagcaaaaggtaagaaaaaaagagaacatgatggagtttggagacaaaaaatgattcacacgtgtggaagacgatagcacacatgccaactattttgtcgtattccttcacaaggcaaataaggacagaatcacaaaacctttttgttgaccgtatatatatatatatatatatattttttttttgaagtacaaaggtgtatgataattatgcatatgatatagaatcctaattttcatgact

SEQ ID NO: 12 - protein, 491 aa, Arabidopsis thaliana
MDTSSSLLFFSFFFFIIIVIFNKINGLRSSPASKKKLNDHHVTSQSHGPKFPHGSLGWPVIGETIEFVSSAYSDRPESFMDKRRLMYGRVFKSHIFGTATIVSTDAEVNRAVLQSDSTAFVPFYPKTVRELMGKSSILLINGSLHRRFHGLVGSFLKSPLLKAQIVRDMHKFLSESMDLWSEDQPVLLQDVSKTVAFKVLAKALISVEKGEDLEELKREFENFISGLMSLPINFPGTQLHRSLQAKKNMVKQVERIIEGKIRKTKNKEEDDVIAKDVVDVLLKDSSEHLTHNLIANNMIDMMIPGHDSVPVLITLAVKFLSDSPAALNLLTEENMKLKSLKELTGEPLYWNDYLSLPFTQKVITETLRMGNVIIGVMRKAMKDVEIKGYVIPKGWCFLAYLRSVHLDKLYYESPYKFNPWRWQERDMNTSSFSPFGGGQRLCPGLDLARLETSVFLHHLVTRFRWIAEEDTIINFPTVHMKNKLPIWIKRI

SEQ ID NO: 13 - DNA, 1770 bp, Arabidopsis thaliana
ctttctctctttctctggtctgtttcaaaacatagtacataataatagagacaacaagagaaagaaaaaacagagtgatctgttttaaaacagagcagaaaacagagtgagatgggagcaatgatggtgatgatgggtcttctcttgatcatcgtgtctctctgttccgctctccttcgatggaatcagatgcgatataccaagaatggtcttcctcctggaaccatgggctggccaatctttggcgaaaccaccgagtttctcaaacaaggccccaacttcatgagaaaccaaagactccgatacgggagtttcttcaaatctcatcttctaggttgtccaacgttaatctcaatggactcagaagtaaacagatacattttaaagaacgaatcaaaaggtttggttcctggttacccacaatcgatgcttgatatacttgggacttgtaacatggctgcggttcacggttcgagccaccggcttatgagaggctcgcttctgtctctcataagctcgaccatgatgagagatcatatcttgcctaaagttgatcacttcatgagaagctatcttgatcagtggaatgagcttgaggttattgatatccaagataagaccaaacatatggcatttttatcttcactgacacaaatcgctgggaacttaagaaaaccatttgttgaagaattcaaaactgcattcttcaagcttgttgttgggactttatccgttccgattgatcttccgggcacaaattatcgttgcggaatccaagcaagaaataacattgataggttgctaagagagctgatgcaagaacgtagagattctggagaaacattcacagacatgttaggttacttgatgaagaaggaaggtaaccgatacccgttaaccgatgaagagataagagaccaagttgtgacgattttgtattcgggttacgaaactgtctctacgacctcaatgatggctcttaagtaccttcatgatcacccaaaagctcttcaagaactaagagctgagcatttggcattcagggaaagaaaacgacaggacgaaccactcggtcttgaggacgtgaagtcaatgaagttcactcgagctgtgatttatgagacatcaagattggcaacgatcgttaatggggtcctaaggaaaactactcgtgacttggaaatcaacggttatttaatcccaaaaggatggagaatttatgtatacacgagggaaattaattacgatgcaaatctttatgaagacccattgatctttaatccatggagatggatgaagaagagcttggagtcacaaaactcatgctttgtgtttggaggtgggacaaggctttgtcctggtaaggaactagggattgtcgagatctcgagctttctccattactttgttacgagatacagatgggaggaaataggaggggatgaattaatggtgtttccgagagtttttgcaccaaaaggcttccatcttaggatttcaccctactaatttctaactttttaaataaaataatatagattttatgcacgcagaggatacagaagacgtatatgaagaaacaagagaagataattataaatttttttttttttttgacacatatttttgttctcatttaagagtcttctggtcatatgtacataaccataagcagcataatcttattatatatctatttctgtataactctatttgaacttgaagtatatctctggctatttcttgatgatgttctactatattaaaccc

SEQ ID NO: 14 - protein, 465 aa, Arabidopsis thaliana
MGAMMVMMGLLLIIVSLCSALLRWNQMRYTKNGLPPGTMGWPIFGETTEFLKQGPNFMRNQRLRYGSFFKSHLLGCPTLISMDSEVNRYILKNESKGLVPGYPQSMLDILGTCNMAAVHGSSHRLMRGSLLSLISSTMMRDHILPKVDHFMRSYLDQWNELEVIDIQDKTKHMAFLSSLTQIAGNLRKPFVEEFKTAFFKLVVGTLSVPIDLPGTNYRCGIQARNNIDRLLRELMQERRDSGETFTDMLGYLMKKEGNRYPLTDEEIRDQVVTILYSGYETVSTTSMMALKYLHDHPKALQELRAEHLAFRERKRQDEPLGLEDVKSMKFTRAVIYETSRLATIVNGVLRKTTRDLEINGYLIPKGWRIYVYTREINYDANLYEDPLIFNPWRWMKKSLESQNSCFVFGGGTRLCPGKELGIVEISSFLHYFVTRYRWEEIGGDELMVFPRVFAPKGFHLRISPY

SEQ ID NO: 15 - DNA, 1784 bp, Arabidopsis thaliana
gttcctatataaacaacgccacacacacccatttagtcccaaccaaagactctttaccatctctttctctctctgtttgaagacatagcacaaaaaaaaaaaaaaagacagagcaaaaaaacacacaaagatgggcataatgatgatgattttgggtcttcttgtgatcattgtttgtttatgtactgctcttctccgatggaaccagatgcgatattctaagaaaggtcttcctcctggaaccatgggctggccaatatttggtgaaacgactgagtttcttaaacaaggaccagatttcatgaaaaaccaaagactaagatatgggagtttcttcaagtctcacattcttggttgcccaacaatagtctcaatggacgcagagttaaacagatacattctaatgaatgaatcgaaaggacttgttgccggttacccgcaatctatgcttgatattctagggacatgcaacatagctgcggttcatggcccgagccaccggctaatgagaggctcgttgctttctttaataagcccaaccatgatgaaagaccatctcttgcctaagattgatgatttcatgagaaactatctttgtggttgggatgatcttgagacagttgatatccaagaaaagaccaaacatatggcatttttatcatcgttgttacaaatagctgagactttgaaaaaaccagaggttgaagaatatagaacagagtttttcaagcttgttgtgggaactctatcggtcccgatcgatatcccgggaacgaattaccgcagtggagtccaagcaagaaacaacatcgataggttattgacagaactgatgcaagaaagaaaagagtctggagaaactttcacagacatgttgggttacttgatgaagaaggaagataaccgatacttgttaaccgataaagagataagagatcaagtggtaacgatcttgtattccggttatgagactgtctctacaacctccatgatggctcttaagtatctccatgatcatccaaaagctcttgaagaactcagaagagaacatttggctataagggagagaaaacgacctgacgaaccgctcactctcgacgatattaaatcgatgaaattcactcgagctgtgatctttgagacatcaagattggcaacgattgttaatggtgtccttaggaaaactactcacgacttagaactcaacggttatttaatcccaaaaggttggagaatttacgtatacacaagagagattaactatgatacatctctttatgaagatccaatgatctttaacccatggagatggatggaaaagagcttagaatcaaagagctatttcttactctttggaggtggagttaggctttgccctggaaaggaactaggaatctcggaagtctcaagcttccttcactactttgttacaaaatatagatgggaagagaatggagaagacaaattaatggtctttccaagagtttctgcaccaaaaggataccatcttaagtgttcaccttactgactagttttgtcctaatattgaaaaatgtgtaaataaatctattaagggtcattttgtagggctaattaacctattttatctattaaatctctcaagatcatagaggagatggataatgtacagagagaaagagagaagaagaaaatggaatatagaaaaaaataaaatatttgaaatgttgagcttagtctcttatcttgtaaatttgtaacccataaatttttacatttcataattacgttctgtgttttttttt

SEQ ID NO: 16 - protein, 465 aa, Arabidopsis thaliana
MGIMMMILGLLVIIVCLCTALLRWNQMRYSKKGLPPGTMGWPIFGETTEFLKQGPDFMKNQRLRYGSFFKSHILGCPTIVSMDAELNRYILMNESKGLVAGYPQSMLDILGTCNIAAVHGPSHRLMRGSLLSLISPTMMKDHLLPKIDDFMRNYLCGWDDLETVDIQEKTKHMAFLSSLLQIAETLKKPEVEEYRTEFFKLVVGTLSVPIDIPGTNYRSGVQARNNIDRLLTELMQERKESGETFTDMLGYLMKKEDNRYLLTDKEIRDQVVTILYSGYETVSTTSMMALKYLHDHPKALEELRREHLAIRERKRPDEPLTLDDIKSMKFTRAVIFETSRLATIVNGVLRKTTHDLELNGYLIPKGWRIYVYTREINYDTSLYEDPMIFNPWRWMEKSLESKSYFLLFGGGVRLCPGKELGISEVSSFLHYFVTKYRWEENGEDKLMVFPRVSAPKGYHLKCSPY

SEQ ID NO: 17 - DNA, 1000 bp, Arabidopsis thaliana
tttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaa

SEQ ID NO: 18 - DNA, 31 bp, Artificial Sequence
  Primer P1
tgtctagaggatcccccttttgggatgggtt

SEQ ID NO: 19 - DNA, 37 bp, Artificial Sequence
  Primer P2
ttcgagctcggtacccggggcgaattcctatgagctg

SEQ ID NO: 20 - DNA, 34 bp, Artificial Sequence
  Primer P3
cataggaattcgccccgggtccccacgtcgaaaa

SEQ ID NO: 21 - DNA, 34 bp, Artificial Sequence
  Primer P4
ttcgagctcggtacccgggcttttgggatgggtt

SEQ ID NO: 22 - DNA, 22 bp, Artificial Sequence
  Primer RD29A FW
gtgagaccctcctctgttttac

SEQ ID NO: 23 - DNA, 21 bp, Artificial Sequence
  Primer SLR RV
cgcaagaccggcaacaggatt

(19) United States
(12) Patent Application Publication
(10) Pub. No.: US 2014/0199760 A1
(43) Pub. Date: Jul. 17, 2014

(54) PLANTS HAVING ENHANCED ABIOTIC STRESS RESISTANCE

(71) Applicants: Julian Geoffrey Northey, Ottawa (CA); Marcus Samuel, Calgary (CA); Peter John McCourt, Toronto (CA)

(72) Inventors: Julian Geoffrey Northey, Ottawa (CA); Marcus Samuel, Calgary (CA); Peter John McCourt, Toronto (CA)

(73) Assignees: UNIVERSITY OF TORONTO, Toronto, ON (CA); FRONTIER AGRI-SCIENCE INC., Ottawa, ON (CA)

(21) Appl. No.: 14/119,018

(22) PCT Filed: May 18, 2012
(86) PCT No.: PCT/CA2012/000480

§ 371 Date: Nov. 20, 2013

Related U.S. Application Data
(60) Provisional application No. 61/488,429, filed on May 20, 2011.
(60) Provisional application No. 61/579,991, filed on Dec. 23, 2011.

(51) Int. Cl. C12N 15/82

(52) U.S. Cl. CPC C12N 15/8273
USPC 435/320.1

(57) ABSTRACT

Means are provided of increasing the growth potential and abiotic stress resistance in plants, characterized by expression of polynucleotides stably integrated into a plant genome or stably incorporated in the plant. Further provided are isolated nucleic acids and their stable inclusion in transgenic plants. The transgenic plants have shown desirable phenotypic characteristics when compared to control plants, for example, improved drought-resistance. Also taught are plants having increased growth potential due to improved abiotic stress resistance.

### FIELD OF INVENTION

The present invention relates to plants that display an enhanced abiotic stress resistance. The invention further relates to plants with an enhanced abiotic stress resistance phenotype.

### BACKGROUND OF THE INVENTION

Most higher plants, which include plants possessing a vascular system, encounter at least transient decreases in relative water content at some stage of their life cycle and, as a result, have evolved a number of desiccation protection mechanisms. If however, the water deficit is prolonged, the effects on the plant's growth and development can be profound. Decreased water content due to drought, heat, cold or salt stresses can irreparably damage plant cells, which in turn limits plant growth and crop productivity in agriculture. Approximately 70% of the genetic yield potential in major crops is lost due to the aforementioned abiotic stresses, with drought and heat having the most detrimental effects. Attempts to improve yield under abiotic stress conditions by plant breeding have been largely unsuccessful, primarily due to the multigenic origin of the adaptive responses (Barkla et al., 1999, Adv Exp Med Biol 464:77-89).

Plants respond to adverse conditions of abiotic stress such as, drought, heat, salinity and cold and biotic stress such as, for example fungal, bacterial or insect with a variety of morphological and physiological changes. Although our understanding of plant tolerance mechanisms to these stresses is fragmentary, the plant hormone abscisic acid (ABA) has been proposed to be an essential mediator between environmental stimulus and plant responses. For example, ABA levels increase in response to water deficits and exogenously applied ABA mimics many of the responses normally induced by water stress. Furthermore, once ABA is synthesized it causes the closure of the leaf stomata, thereby decreasing water loss through transpiration.

The identification of genes that transduce ABA into a cellular response, for example by affecting ABA levels and/or sensitivity may lead to the possibility of exploiting these regulators to enhance desiccation tolerance in crop species. In principle, these ABA signaling genes can be coupled with the appropriate controlling elements to allow optimal plant growth and development. Thus, not only would these genes allow the genetic tailoring of crops to withstand transitory environmental stresses, they would also broaden the types of environments in which traditional crops can be grown.

Brassinosteroids (BRs) are polyhydroxylated steroid hormones that regulate plant growth and development. Brassinolide is typically the most active BR and is the endpoint of the biosynthetic pathway. BRs are synthesized from campesterol, which is derived from the plant sterol precursor, cycloartenol. Campesterol is first converted to campestanol in multiple steps which involve the enzyme steroid 5-alpha-reductase. Campestanol is eventually converted to castasterone, which also typically displays bioactivity, through either of two linked pathways, the early and late C-6 oxidation pathways. All enzymes discovered to date that are involved in the conversion of campestanol to brassinolide are cytochrome P450 monooxygenases.

Several studies have demonstrated the ability of BRs to increase the yields of crop plants. For example, brassinolide has been found to increase bean crop yield by approximately 45%, and similar increases in yield have been observed for rice, wheat, barley etc. Addition of bioactive BRs have also promoted potato tuber growth and increased its resistance to infections. In addition to the growth promoting capabilities of bioactive BRs, applied BR can also significantly increase the yield of crops grown under conditions of stress.

However, little further study has been conducted in the area of the mechanism by which BRs affect crop yield and very little work has been done on the effects of inhibiting BRs (biosynthesis and/or signaling) in plant cells.

### SUMMARY OF THE INVENTION

The present invention thus provides an isolated nucleic acid comprising a polynucleotide sequence that encodes a polypeptide having an amino acid sequence with at least 40% percent identity to the amino acid sequence set forth in SEQ ID NO:2, SEQ ID NO:4, SEQ ID NO:6 SEQ ID NO:8, SEQ ID NO:10, SEQ ID NO:12, SEQ ID NO:14, or SEQ ID NO:16.

The present invention further provides a nucleic acid construct comprising a promoter operably linked to a nucleic acid that ultimately inhibits the polynucleotide expression or polypeptide function selected from the group consisting of a polynucleotide as defined in SEQ ID NO:1, SEQ ID NO:3, SEQ ID NO:5, SEQ ID NO:7 SEQ ID NO:9, SEQ ID NO:11, SEQ ID NO:13, or SEQ ID NO:15; a polynucleotide encoding a polypeptide as defined in SEQ ID NO:2, SEQ ID NO:4, SEQ ID NO:6 SEQ ID NO:8, SEQ ID NO:10, SEQ ID NO:12, SEQ ID NO:14, or SEQ ID NO:16; a polynucleotide having at least 40% sequence identity to a polynucleotide as defined in SEQ ID NO:1, SEQ ID NO:3, SEQ ID NO:5, SEQ ID NO:7 SEQ ID NO:9, SEQ ID NO:11, SEQ ID NO:13, or SEQ ID NO:15; and a polynucleotide encoding a polypeptide having at least 40% sequence identity to a polypeptide as defined in SEQ ID NO:2, SEQ ID NO:4, SEQ ID NO:6 SEQ ID NO:8, SEQ ID NO:10, SEQ ID NO:12, SEQ ID NO:14, or SEQ ID NO:16.

A DNA based molecule, for carrying the said nucleic acid construct of the present invention is also provided, including but not limited to plasmids and vectors.

Transgenic plants, as well as the cells and seeds thereof, and transgenic tissue cultures are also provided, comprising the nucleic acid of the present invention. The present invention also provides a transgenic plant regenerated and comprising the plant cell or the tissue culture of the present invention.

A plant comprising the present nucleic acid is also provided in which the nucleic acid comprises an allele that results in increased growth, increased abiotic stress tolerance or increased water use efficiency under stress conditions over wild type varieties of the plant or plants lacking the allele.

In addition, a method is described herein for increasing growth or abiotic stress tolerance in a plant. The method comprises inhibiting the function in said plant of at least one polypeptide comprising an amino acid sequence as set forth in SEQ ID NO:2, SEQ ID NO:4, SEQ ID NO:6 SEQ ID NO:8, SEQ ID NO:10, SEQ ID NO:12, SEQ ID NO:14, or SEQ ID NO:16, or a polypeptide comprising an amino acid sequence with at least 40% percent identity thereto. In certain preferred embodiments, the amino acid sequence has from 80 percent to 99 percent identity, more preferably from 95 to 99 percent identity to SEQ ID NO:2, SEQ ID NO:4, SEQ ID NO:6 SEQ ID NO:8, SEQ ID NO:10, SEQ ID NO:12, SEQ ID NO:14, or SEQ ID NO:16.

Without wishing to be limiting in any way, the function of the polypeptide may be inhibited by chemical means, by mutagenesis or disruption of the gene(s) encoding the polypeptide(s), through disruption of the translational mechanisms for expression of the polypeptide(s), or by other means.

A transgenic plant, as well as the cells and seeds of a transgenic plant produced according to the method are also provided.

### BRIEF DESCRIPTION OF THE DRAWINGS

These and other features of the invention will become more apparent from the following description in which reference is made to the following appended drawings:

FIG. 1. RT-PCR analysis of CYP85A2 in wild-type Columbia and cyp85a2. Total RNA was extracted from 7 day old seedlings (1 mg).

FIG. 2. (A) Soil water content of wild-type Columbia (red), era1-2 (green), and line cyp85a2 (blue) during a drought treatment. All samples had a starting weight of 280 g including water and drought was induced by withholding water once the plants began to flower. Error bars represent standard error (n=8). (B) Water loss during the first 9 days of drought treatment divided by the final shoot dry weight (SDW) in wild-type Columbia, era1-2, and line cyp85a2. Error bars represent standard error (n=6). (C) Cold stress treatment. (D) Water loss during the first 9 days of drought treatment divided by the final shoot dry weight (SDW) in wild-type Columbia, line D4-1, D4-2, and D4-3 Error bars represent standard error (n=6) (E) Water loss during the first 4 days of drought treatment divided by the final shoot dry weight (SDW) in canola (n=8), soybean (n=8), and corn (n=4) under untreated conditions (blue) and with the addition of the chemical (red). Canola and soybean were treated with 10 uM and corn with 20 uM of the chemical (final concentration, added to soil). Error bars represent standard deviation.

### DETAILED DESCRIPTION

The present invention relates to increasing the growth potential and abiotic resistance in plants, characterized by expression of polynucleotides stably integrated into a plant genome. The invention further relates to isolated nucleic acids and their inclusion in transgenic plants. The transgenic plants provided herein have shown desirable phenotypic characteristics when compared to control plants, for example, improved drought-resistance. The present invention also relates to plants having increased growth potential due to improved abiotic stress resistance.

This invention relates to isolated nucleic acids which encode BR-biosynthetic enzymes comprising SEQ ID NO:1, SEQ ID NO:3, SEQ ID NO:5, SEQ ID NO:7 SEQ ID NO:9, SEQ ID NO:11, SEQ ID NO:13, or SEQ ID NO:15. Nucleic acids also included in the present invention are such hybridizing sequences which encode functional equivalents or fragments thereof of SEQ ID NO:1, SEQ ID NO:3, SEQ ID NO:5, SEQ ID NO:7 SEQ ID NO:9, SEQ ID NO:11, SEQ ID NO:13, or SEQ ID NO:15. The present invention also relates to a method for enhancing the abiotic stress resistance of plants by using inhibitors of products encoded by these nucleic acids. Further, the invention relates to the control of regulatory functions in photosynthetic organisms; for example, in the control of growth habit, flowering, seed production, seed germination, and senescence in such organisms.

This invention also relates to a method for enhancing the abiotic stress resistance of plants by means of alterations in isolated or recombinant nucleic acids encoding proteins provided in SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18, or fragment thereof or its functional equivalent. Nucleic acids which hybridize to the aforementioned BR-biosynthetic genes (SEQ ID NO:1, SEQ ID NO:3, SEQ ID NO:5, SEQ ID NO:7 SEQ ID NO:9, SEQ ID NO:11, SEQ ID NO:13, or SEQ ID NO:15) are also encompassed by this invention when such hybridizing sequences encode the functional equivalent or fragment thereof of the said proteins. The present invention also relates to a method for enhancing the abiotic stress resistance of plants through the genetic manipulation of the aforementioned BR-biosynthetic genes and their functional equivalents to improve stress resistance in crop plants. Loss of BR-biosynthetic gene function confers enhanced abiotic stress resistance at the level of the mature plant. The nature of a BR-biosynthetic mutant with loss of BR enzymatic activity, for example, demonstrates that inhibition of BR-biosynthesis and BR signaling enhances ABA responses in a plant, thereby enhancing abiotic stress resistance.

Further, this invention relates to inhibition of senescence in photosynthetic organisms through inhibition of BR-biosynthesis. The resulting photosynthetic organisms stay green and tissue viability is maintained for a longer period of time. Thus, methods to provide greener plants and a reduction in senescence are part of this invention.

The invention also provides methods of producing a transgenic plant, which has an altered phenotype such as increased resistance to abiotic stress, delayed senescence or increased ABA sensitivity by introducing into a plant cell a compound that inhibits a polynucleotide or polypeptide involved in BR-biosynthesis. In one aspect the compound inhibits BR-biosynthesis gene expression or activity. The compound could be, for example, an anti-sense BR-biosynthetic nucleic acid or a BR-biosynthetic double stranded RNA-inhibition hairpin nucleic acid. In some aspects the nucleic acid is operably linked to a promoter such as, for example, a constitutive promoter, an ABA inducible promoter, an abiotic stress inducible promoter (such as but not limited to a drought inducible promoter), tissue specific promoters or a guard cell-specific promoter.

Also included in the invention are the plants produced by the methods of the invention and the seed produced by the plants which produce a plant that has an altered phenotype.

All publications, patent applications, patents, and other references mentioned herein are incorporated by reference in their entirety.

Unless otherwise indicated, all technical and scientific terms used herein have the same meaning as they would to one skilled in the art of the present invention. Practitioners are particularly directed to Sambrook et al., Molecular Cloning: A Laboratory Manual (Second Edition), Cold Spring Harbor Press, Plainview, N.Y., 1989, and Ausubel F M et al. Current Protocols in Molecular Biology, John Wiley & Sons, New York, N.Y., 1993.

As used herein, the term “gene expression” refers to the process by which a polypeptide is produced based on the nucleic acid sequence of a gene. The process includes both transcription and translation; accordingly, “expression” may refer to either a polynucleotide or polypeptide sequence, or both. Sometimes, expression of a polynucleotide sequence will not lead to protein translation. “Overexpression” refers to increased expression of a polynucleotide and/or polypeptide sequence relative to its expression in a wild-type or other non-transgenic plant and may relate to a naturally-occurring or non-naturally occurring sequence. “Ectopic expression” refers to expression at a time, place, and/or increased level that does not naturally occur in the non-altered or wild-type plant. “Under-expression” refers to decreased expression of a polynucleotide and/or polypeptide sequence, generally of an endogenous gene, relative to its expression in a wild-type plant. The terms “mis-expression” and “altered expression” encompass over-expression, under-expression, and ectopic expression.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

As used herein, the terms “native” and “wild-type” relative to a given plant trait or phenotype refers to the form in which that trait or phenotype is found in the same variety of plant in nature.

As used herein, the term “modified” regarding a plant trait, refers to a change in the phenotype of a transgenic plant relative to the similar non-transgenic plant. An “interesting phenotype (trait)” with reference to a transgenic plant refers to an observable or measurable phenotype demonstrated by a T1 and/or subsequent generation plant, which is not displayed by the corresponding non-transgenic (i.e., a genotypically similar plant that has been raised or assayed under similar conditions).

An “altered drought-resistant phenotype” refers to detectable change in the ability of a genetically modified plant to withstand low-water conditions compared to the similar, but non-modified plant. In general, improved or increased drought-resistant phenotypes (i.e., ability to a plant to survive in low-water conditions that would normally be deleterious to a plant) are of interest.

As used herein, the term “T1” refers to the generation of plants from the seed of TO plants. The T1 generation is the first set of transformed plants that can be selected by application of a selection agent, e.g., an antibiotic or herbicide, for which the transgenic plant contains the corresponding resistance gene. The term “T2” refers to the generation of plants by self-fertilization of the flowers of T1 plants, previously selected as being transgenic.

As used herein, the term “plant part” is meant to include a portion of a plant capable of producing a regenerated plant and includes any plant organ or tissue, including, without limitation, seeds, embryos, meristematic regions, callus tissue, leaves, roots, shoots, gametophytes, sporophytes, pollen, and microspores and the like. Preferable plant parts include roots and shoots and meristematic portions thereof. Plant cells can be obtained from any plant organ or tissue and cultures prepared therefrom. Transgenic plants can be regenerated from any of these plant parts, including tissue culture or protoplasts, and also from explants. Methods will vary according to the species of plant. The class of plants which can be used in the methods of the present invention is generally as broad as the class of higher plants amenable to transformation techniques, including both monocotyledenous and dicotyledenous plants.

### BR-BioSig Nucleic Acids and Polypeptides

Arabidopsis DWF1, DET2, DWF4, CPD, ROT3, CYP90D1, CYP85A1 and CYP85A2 nucleic acid (cDNA) sequence is provided in SEQ ID NO:1, SEQ ID NO:3, SEQ ID NO:5, SEQ ID NO:7 SEQ ID NO:9, SEQ ID NO:11, SEQ ID NO:13, and SEQ ID NO:15 and in NCBI gene id 821519, 818383, 824229, 830453, 829790, 820582, 833889 and 822709 respectively. The corresponding protein sequence is provided in SEQ ID NO:2, SEQ ID NO:4, SEQ ID NO:6, SEQ ID NO:8, SEQ ID NO:10, SEQ ID NO:12, SEQ ID NO:14, and SEQ ID NO:16. Their TAIR designations are AT3G19820, AT2G38050, AT3G50660, AT5G05690, AT4G36380, AT3G13730, AT5G38970 and AT3G30180 respectively.

As used herein, the term “BR-BioSig” polypeptide refers to a full-length protein or a fragment, derivative, variant, or ortholog thereof that is functionally active, meaning that the protein fragment, derivative, or ortholog exhibits one or more or the functional activities associated with the polypeptide of SEQ ID NO:2, SEQ ID NO:4, SEQ ID NO:6, SEQ ID NO:8, SEQ ID NO:10, SEQ ID NO:12, SEQ ID NO:14, or SEQ ID NO:16. In one preferred embodiment, inhibition or down-regulation of a functionally active BR-BioSig polypeptide causes an altered drought-resistant phenotype in a plant. In a further preferred embodiment, a dominant-negative mutation or mis-expression of the functionally active BR-BioSig polypeptide causes improved drought-resistance. In another embodiment, a functionally active BR-BioSig polypeptide is capable of rescuing defective or deficient endogenous BR-BioSig activity when expressed in a plant or in plant cells; the rescuing polypeptide may be from the same or from a different species as that with defective activity. Functionally active variants of full-length BR-BioSig polypeptides or fragments thereof include polypeptides with amino acid insertions, deletions, or substitutions that retain one or more of the biological properties associated with the full length BR-BioSig polypeptide. In some cases, variants are generated that change the post-translational processing of a BR-BioSig polypeptide. For instance, variants may have altered protein transport or protein localization characteristics or altered protein half-life compared to the native polypeptide.

As used herein, the term “BR-BioSig” nucleic acid encompasses nucleic acids with the sequence provided in or complementary to the sequence provided in SEQ ID NO:1, SEQ ID NO:3, SEQ ID NO:5, SEQ ID NO:7 SEQ ID NO:9, SEQ ID NO:11, SEQ ID NO:13, or SEQ ID NO:15, as well as functionally active fragments, derivatives, or orthologs thereof. A BR-BioSig nucleic acid of this invention may be DNA, derived from genomic DNA or cDNA, or RNA. In one preferred embodiment, inhibition or down-regulation of a functionally active BR-BioSig nucleic acid causes an altered drought-resistant phenotype in a plant.

In one embodiment, a functionally active BR-BioSig nucleic acid encodes or is complementary to a nucleic acid that encodes a functionally active BR-BioSig polypeptide. Included within this definition is genomic DNA that serves as a template for a primary RNA transcript, that is, an mRNA precursor that requires processing, such as splicing, before encoding the functionally active BR-BioSig polypeptide. A BR-BioSig nucleic acid can include other non-coding sequences, which may or may not be transcribed; such sequences include 5′ and 3′ UTRs, polyadenylation signals and regulatory sequences that control gene expression, among others, as are known in the art. Some polypeptides require processing events, such as proteolytic cleavage, covalent modification, etc., in order to become fully active. Accordingly, functionally active nucleic acids may encode the mature or the pre-processed BR-BioSig polypeptide, or an intermediate form. A BR-BioSig polynucleotide can also include heterologous coding sequences, for example, sequences that encode a marker included to facilitate the purification of the fused polypeptide, or a transformation marker.

In another embodiment, a functionally active BR-BioSig nucleic acid or fragment thereof is capable of being used in the generation of loss-of-function BR-BioSig phenotypes, for instance, via antisense suppression, co-suppression or post-transcriptional gene silencing (PGTS).

In one preferred embodiment, a BR-BioSig nucleic acid used in the methods of this invention comprises a nucleic acid sequence that encodes or is complementary to a sequence that encodes a BR-BioSig polypeptide having at least 40%, 45%, 50%, 60%, 70%, 75%, 80%, 85%, 90%, 95% or more sequence identity to the polypeptide sequence presented in SEQ ID NO:2, SEQ ID NO:4, SEQ ID NO:6, SEQ ID NO:8, SEQ ID NO:10, SEQ ID NO:12, SEQ ID NO:14, or SEQ ID NO:16.

In another embodiment a BR-BioSig polypeptide of the invention comprises a polypeptide sequence with at least 40% or 50% identity to the BR-BioSig polypeptide sequence of SEQ ID NO:2, SEQ ID NO:4, SEQ ID NO:6, SEQ ID NO:8, SEQ ID NO:10, SEQ ID NO:12, SEQ ID NO:14, or SEQ ID NO:16, and may have at least 60%, 70%, 80%, 85%, 90% or 95% or more sequence identity to the BR-BioSig polypeptide sequence of SEQ ID NO:2, SEQ ID NO:4, SEQ ID NO:6, SEQ ID NO:8, SEQ ID NO:10, SEQ ID NO:12, SEQ ID NO:14, or SEQ ID NO:16. In another embodiment, a BR-BioSig polypeptide comprises a polypeptide sequence with at least 40%, 50%, 60%, 70%, 80%, 85%, 90% or 95% or more sequence identity to a functionally active fragment of the polypeptide presented in SEQ ID NO:2, SEQ ID NO:4, SEQ ID NO:6, SEQ ID NO:8, SEQ ID NO:10, SEQ ID NO:12, SEQ ID NO:14, or SEQ ID NO:16, such as a P450 domain or other necessary functional domain. In yet another embodiment, a BR-BioSig polypeptide comprises a polypeptide sequence with at least 40%, 50%, 60%, 70%, 80%, or 90% identity to the polypeptide sequence of SEQ ID NO:2, SEQ ID NO:4, SEQ ID NO:6, SEQ ID NO:8, SEQ ID NO:10, SEQ ID NO:12, SEQ ID NO:14, or SEQ ID NO:16 over its entire length and comprises a catalytic domain.

In another aspect, a BR-BioSig polynucleotide sequence is at least 40% to 50% identical over its entire length to the BR-BioSig nucleic acid sequence presented as SEQ ID NO:1, SEQ ID NO:3, SEQ ID NO:5, SEQ ID NO:7 SEQ ID NO:9, SEQ ID NO:11, SEQ ID NO:13, or SEQ ID NO:15, or nucleic acid sequences that are complementary to such a BR-BioSig sequence, and may comprise at least 60%, 70%, 80%, 85%, 90% or 95% or more sequence identity to the BR-BioSig sequence presented as SEQ ID NO:1, SEQ ID NO:3, SEQ ID NO:5, SEQ ID NO:7 SEQ ID NO:9, SEQ ID NO:11, SEQ ID NO:13, or SEQ ID NO:15, or a functionally active fragment thereof, or complementary sequences.

As used herein, “percent (%) sequence identity” with respect to a specified subject sequence, or a specified portion thereof, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0 with search parameters set to default values (Altschul et al., J. Mol. Biol. (1990) 215:403-410; website at blast.wustl.edu/blast/README.html).

The HSPS and HSPS2 parameters are dynamic values and are established by the WU-BLAST program itself depending upon the composition of the particular sequence and composition of the particular database against which the sequence of interest is being searched. A “% identity value” is determined by the number of matching identical nucleotides or amino acids divided by the sequence length for which the percent identity is being reported. “Percent (%) amino acid sequence similarity” is determined by the same calculation as used for determining % amino acid sequence identity, but including conservative amino acid substitutions in addition to identical amino acids in the computation. A conservative amino acid substitution is one in which an amino acid is substituted for another amino acid having similar properties such that the folding or activity of the protein is not significantly affected. Aromatic amino acids that can be substituted for each other include phenylalanine, tryptophan, and tyrosine. Interchangeable hydrophobic amino acids include leucine, isoleucine, methionine, and valine. Interchangeable polar amino acids include glutamine and asparagines. Interchangeable basic amino acids include arginine, lysine and histidine. Interchangeable acidic amino acids include aspartic acid and glutamic acid. Finally, interchangeable small amino acids include alanine, serine, threonine, cysteine and glycine.

Derivative nucleic acid molecules of the subject nucleic acid molecules include sequences that hybridize to the nucleic acid sequence of SEQ ID NO:1, SEQ ID NO:3, SEQ ID NO:5, SEQ ID NO:7 SEQ ID NO:9, SEQ ID NO:11, SEQ ID NO:13, SEQ ID NO:15. The stringency of hybridization can be controlled by temperature, ionic strength, pH, and the presence of denaturing agents such as formamide during hybridization and washing. Conditions routinely used would be well known to those in the art and are encompassed in the present invention (see, e.g., Current Protocol in Molecular Biology, Vol. I, Chap. 2.10, John Wiley & Sons, Publishers (1994); Sambrook et al., supra).

In some embodiments a nucleic acid molecule of the present invention is capable of hybridizing to a nucleic acid molecule containing the nucleotide sequence of SEQ ID NO: 1 under stringent hybridization conditions that comprise: prehybridization of filters containing nucleic acid for 8 hours to overnight at 65° C. in a solution comprising 6× single strength citrate (SSC) (1×SSC is 0.15 M NaCI, 0.015 M Na citrate; pH 7.0), 5×Denhardt's solution, 0.05% sodium pyrophosphate and 100 ug/ml herring sperm DNA; hybridization for 18-20 hours at 65° C. in a solution containing 6×SSC, 1×Denhardt's solution, 100 ug/ml yeast tRNA and 0.05% sodium pyrophosphate; and washing of filters at 65° C. for 1 h in a solution containing 0.2×SSC and 0.1% SDS (sodium dodecylsulfate). In other embodiments, moderately stringent hybridization conditions are used that comprise: pretreatment of filters containing nucleic acid for 6 h at 40° C. in a solution containing 35% formamide, 5×SSC, 50 mM Tris HCI (pH 7.5), 5 mM EDTA, 0.1% PVP, 0.1% Ficoll, 1% BSA, and 500 ug/ml denatured salmon sperm DNA; hybridization for 18-20 h at 40° C. in a solution containing 35% formamide, 5×SSC, 50 mM Tris-HCI (pH 7.5), 5 mM EDTA, 0.02% PVP, 0.02% Ficoll, 0.2% BSA, 100 ug/ml salmon sperm DNA, and 10% (wt/vol) dextran sulfate; followed by washing twice for 1 hour at 55° C. in a solution containing 2×SSC and 0.1% SDS. Alternatively, low stringency conditions can be used that comprise: incubation for 8 hours to overnight at 37° C. in a solution comprising 20% formamide, 5×SSC, 50 mM sodium phosphate (pH 7.6), 5×Denhardt's solution, 10% dextran sulfate, and 20 ug/ml denatured sheared salmon sperm DNA; hybridization in the same buffer for 18 to 20 hours; and washing of filters in 1×SSC at about 37° C. for 1 hour.

As a result of the degeneracy of the genetic code, a number of polynucleotide sequences encoding a BR-BioSig polypeptide can be produced. For example, codons may be selected to increase the rate at which expression of the polypeptide occurs in a particular host species, in accordance with the optimum codon usage dictated by the particular host organism (see, e.g., Nakamura Y et al, Nucleic Acids Res (1999) 27:292). Such sequence variants may be used in the methods of this invention.

The methods of the present invention may use orthologs of the Arabidopsis BR-BioSig. Methods of identifying the orthologs in other plant species are known in the art. Normally, orthologs in different species retain the same function, due to presence of one or more protein motifs and/or 3-dimensional structures. In evolution, when a gene duplication event follows speciation, a single gene in one species, such as Arabidopsis, may correspond to multiple genes, or paralogs, in another. As used herein, the term “orthologs” encompasses paralogs. When sequence data is available for a particular plant species, orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:12041210). Programs for multiple sequence alignment, such as CLUSTAL (Thompson J D et al, Nucleic Acids Res (1994) 22:4673-4680) may be used to highlight conserved regions and/or residues of orthologous proteins and to generate phylogenetic trees. In a phylogenetic tree representing multiple homologous sequences from diverse species for example those, retrieved through BLAST analysis, orthologous sequences from two species generally appear closest on the tree with respect to all other sequences from these two species. Structural threading or other analysis of protein folding for example by using software by ProCeryon, Biosciences, Salzburg, Austria, may also identify potential orthologs. Nucleic acid hybridization methods may also be used to find orthologous genes and are preferred when sequence data are not available. Degenerate PCR and screening of cDNA or genomic DNA libraries are common methods for finding related gene sequences and are well known in the art (see, e.g., Sambrook, supra; Dieffenbach C and Dveksler G (Eds.) PCR Primer: A Laboratory Manual, Cold Spring Harbor Laboratory Press, NY, 1989). For instance, methods for generating a cDNA library from the plant species of interest and probing the library with partially homologous gene probes are described in Sambrook et al.

A highly conserved portion of the Arabidopsis BR-BioSig coding sequence may be used as a probe. BR-BioSig ortholog nucleic acids may hybridize to the nucleic acid of SEQ ID NO:1, SEQ ID NO:3, SEQ ID NO:5, SEQ ID NO:7 SEQ ID NO:9, SEQ ID NO:11, SEQ ID NO:13, or SEQ ID NO:15 under high, moderate, or low stringency conditions. After amplification or isolation of a segment of a putative ortholog, that segment may be cloned and sequenced by standard techniques and utilized as a probe to isolate a complete cDNA or genomic clone. Alternatively, it is possible to initiate an EST project to generate a database of sequence information for the plant species of interest.

In another approach, antibodies that specifically bind known BR-BioSig polypeptides are used for ortholog isolation. Western blot analysis can determine that a BR-BioSig ortholog (i.e., an orthologous protein) is present in a crude extract of a particular plant species. When reactivity is observed, the sequence encoding the candidate ortholog may be isolated by screening expression libraries that represent the particular plant species. Expression libraries can be constructed in a variety of commercially available vectors, including lambda gt 11, as described in Sambrook, et al., supra. Once the candidate ortholog(s) are identified by any of these means, candidate orthologous sequence are used as bait (the “query”) for the reverse BLAST against sequences from Arabidopsis or other species in which BR-BioSig nucleic acid and/or polypeptide sequences have been identified.

BR-BioSig nucleic acids and polypeptides may be obtained using any available method. For instance, techniques for isolating cDNA or genomic DNA sequences of interest by screening DNA libraries or by using polymerase chain reaction (PCR), as previously described, are well known in the art. Alternatively, nucleic acid sequence may be synthesized. Any known method, such as site directed mutagenesis (Kunkel T A et al., Methods Enzymol. (1991) 204:125-39), may be used to introduce desired changes into a cloned nucleic acid.

In general, the methods of the invention involve incorporating the desired form of the BR-BioSig nucleic acid into a plant expression vector for transformation of in plant cells, and subsequent inhibition of the BR-BioSig polypeptide in the host plant.

An isolated BR-BioSig nucleic acid molecule is other than in the form or setting in which it is found in nature and is identified and separated from least one contaminant nucleic acid molecule with which it is ordinarily associated in the natural source of the BR-BioSig nucleic acid. However, an isolated BR-BioSig nucleic acid molecule includes BR-BioSig nucleic acid molecules contained in cells that ordinarily express BR-BioSig where, for example, the nucleic acid molecule is in a chromosomal location different from that of natural cells.

Generation of Genetically Modified Plants with Abiotic Stress Resistance.

BR-BioSig nucleic acids and polypeptides may be used in the generation of genetically modified plants having a modified, preferably an improved drought-resistant phenotype. Such plants may further display increased resistance to other abiotic stresses, in particular salt-stress and freezing, as responses to these stresses and drought stress are mediated by ABA (Thomashow, 1999 Annu. Revl Plant Physiol. Plant Mol. Biol 50: 571; Cushman and Bohnert, 2000, Curro Opin. Plant BioI. 3: 117; Kang et al. 2002, Plant Cell 14:343-357; Quesada et al. 2000, Genetics 154: 421; Kasuga et al. 1999, Nature Biotech. 17: 287-291).

The methods described herein are generally applicable to all plants. Drought-resistance is an important trait in almost any agricultural crop; most major agricultural crops, including corn, wheat, soybeans, cotton, alfalfa, sugar beets, onions, tomatoes, and beans, are all susceptible to drought stress. Although the specific inhibition of BR-BioSig functions is carried out in Arabidopsis, BR-BioSig genes, or an ortholog, variant or fragment thereof, may be inhibited in any type of plant. The potential use of the present invention could be applied to all types of plants and other photosynthetic organisms, including, but not limited to: angiosperms, including monocots and dicots, gymnosperms, spore-bearing or vegetatively-reproducing plants and the algae, including the cyanophyta (blue-green algae). More preferably, the present invention may thus be directed to fruit- and vegetable-bearing plants such as tomato (Lycopersicum esculentum), eggplant, pea, alfalfa (Medicago sativa), potato, manihot, solanaceous plants, plants used in the cut flower industry including Vicia species, tagetes, Salix species, grain-producing plants such as maize, wheat, rye, oat, triticale, rice, millet, sorghum, barley, oil-producing plants such as, rapeseed, including canola, sunflower, oil palm, coconut, nut-producing plants like peanut, other commercially-valuable crops including sugar beet, coffee, cacao, tea, soybean (Glycine max), cotton (Gossypium), flax (Linum usitatissimumi), tobacco (Nicotiana), pepper, perennial grasses such as sugarcane and turfgrass (Poaceae family) and other forage crops, as well as conifers, evergreens and additional gymnosperm species.

The skilled artisan will recognize that a wide variety of transformation techniques exist in the art and new techniques are continually becoming available. Any technique that is suitable for the target host plant can be employed within the scope of the present invention. For example, the constructs can be introduced in a variety of forms including, but not limited to as a strand of DNA, in a plasmid, or in an artificial chromosome (Halpin C (2005) Plant Biotechnol J 3: 141-155; Mach et al., U.S. Pat. Nos. 7,227,057, 7,226,782; Copenhaver et al., U.S. Pat. No. 7,193,128), or through the use of specifically engineered zinc-finger proteins (Shukla et al., (2009) Nature 459, 437-441). The introduction of the constructs into the target plant cells can be accomplished by a variety of techniques, including, but not limited to Agrobacterium mediated transformation, electroporation, microinjection, microprojectile bombardment calcium phosphate-DNA co-precipitation or liposome-mediated transformation of a heterologous nucleic acid. The transformation of the plant is preferably permanent, that is, by integration of the introduced expression constructs into the host plant genome, so that the introduced constructs are passed onto successive plant generations. Depending upon the intended use, a heterologous nucleic acid construct comprising a BR-BioSig polynucleotide may encode the entire protein or a portion thereof.

In one embodiment, binary Ti-based vector systems may be used to transfer polynucleotides. Standard Agrobacterium binary vectors are known to those of skill in the art, and many are commercially available (e.g., pBI121 Clontech Laboratories).

The optimal procedure for transformation of plants with Agrobacterium vectors will vary with the type of plant being transformed. Exemplary methods for Agrobacterium mediated transformation include transformation of explants of hypocotyl, shoot tip, stem or leaf tissue, derived from sterile seedlings and/or plantlets. Such transformed plants may be reproduced sexually, or by cell or tissue culture. Agrobacterium transformation has been previously described for a large number of different types of plants and methods for such transformation may be found in the scientific literature.

### Regeneration of Transformants

The development or regeneration of plants from either single plant protoplasts or various explants is well known in the art (Weissbach, A., and Weissbach, H., eds (1988). “Methods for Plant Molecular Biology.” Academic Press, San Diego). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous DNA based construct introduced by Agrobacterium from leaf explants can be achieved by methods well known in the art such as described by (Horsch R. B., Fry J. E., Hoffmann N, Wallroth M, Eichholtz D, Rogers S. G., Fraley R. T. (1985) Science 227:1229-1231). In this procedure, transformants are cultured in the presence of a selection agent and in a medium that induces the regeneration of shoots in the plant strain being transformed as described by (FRALEY, R. T., ROGERS, S. G., HORSCH, R. B., SANDERS, P. R., FLICK, J. S., ADAMS, S. P., BITTNER, M. L., BRAND, L. A., FINK, C. L., FRY, J. S., GALLUPPI, G. R., GOLDBERG, S. B., HOFFMANN, N. L. and WOO, S. C. (1983). Proc. Natl. Acad. Sci. USA 80: 4803-4807). In particular, U.S. Pat. No. 5,349,124, the specification of which is incorporated herein by reference, details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art. Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired DNA based construct is cultivated using methods well known to one skilled in the art. A preferred transgenic plant is an independent segregant and can transmit the gene and its activity to its progeny. A more preferred transgenic plant is homozygous for the gene, and transmits that gene to all of its offspring on sexual mating. Seed from a transgenic plant may be grown in the field or greenhouse, and resulting sexually mature transgenic plants are self-pollinated to generate true breeding plants. The progeny from these plants become true breeding lines that are evaluated for increased expression of the transgene.

The methods of this invention can also be used with in planta or seed transformation techniques which do not require culture or regeneration. Examples of these techniques are described in Bechtold, N., et al. (1993) CR Acad. Sci. Paris/Life Sciences 316:118-93; Chang, S. S., et al. (1990) Abstracts of the Fourth International Conference on Arabidopsis Research, Vienna, p. 28; Feldmann, K. A. and Marks, D. M (1987) Mol. Gen. Genet. 208:1-9; Ledoux, L., et al. (1985) Arabidopsis In Serv. 22:1-1 1; Feldmann, K. A (1992) In: Methods in Arabidopsis Research (Eds. Koncz, c., Chua, N-H, Schell, J.) pp. 274-289; Chee, et al., U.S. Pat. No. 5,376,543, all of which are incorporated herein by reference.

To aid in identification of transformed plant cells, the constructs of this invention are further manipulated to include genes coding for plant selectable markers. Useful selectable markers include enzymes which provide for resistance to an antibiotic such as gentamycin, hygromycin, kanamycin, or the like. Similarly, enzymes providing for production of a compound identifiable by color change such as GUS (˜-glucuronidase), or by luminescence, such as luciferase, are useful. For example, antisense BR-BioSig can be produced by integrating a complement of any of the BR-BioSig genes linked to DNA comprising the SEQ ID NO:19 promoter into the genome of a virus that enters the host cells. By infection of the host cells, the components of a system that permit the transcription of the antisense, are then present in the host cells. When cells or protoplasts containing the antisense gene driven by a promoter of the present invention are obtained, the cells or protoplasts are regenerated into whole plants. The transformed cells are then cultivated under conditions appropriate for the regeneration of plants, resulting in production of transgenic plants. Choice of methodology for the regeneration step is not critical, with suitable protocols being available for many varieties of plants, tissues and other photosynthetic organisms. See, e.g., Gelvin S. B. and Schilperoort R. A, eds. Plant Molecular Biology Manual, Second Edition, Suppl. I (1995) Kluwer Academic Publishers, Boston Mass., U.S.A. Transgenic plants carrying the construct are examined for the desired phenotype using a variety of methods including but not limited to an appropriate phenotypic marker, such as antibiotic resistance or herbicide resistance as described supra, or visual observation of their growth compared to the growth of the naturally-occurring plants under the same conditions.

### Transfer by Plant Breeding

Alternatively, once a single transformed plant has been obtained by the foregoing recombinant DNA method, conventional plant breeding methods can be used to transfer the gene and associated regulatory sequences via crossing and backcrossing. Such intermediate methods will comprise the further steps of: (1) sexually crossing the transgenic plant with a plant from a second taxon; (2) recovering reproductive material from the progeny of the cross; and (3) growing transgenic plants from the reproductive material. Where desirable or necessary, the agronomic characteristics of the second taxon can be substantially preserved by expanding this method to include the further steps of repetitively: (1) backcrossing the transgenic progeny with non-transgenic plants from the second taxon; and (2) selecting for expression of an associated marker gene among the progeny of the backcross, until the desired percentage of the characteristics of the second taxon are present in the progeny along with the gene or genes imparting marker gene trait. By the term “taxon” herein is meant a unit of botanical classification. It thus includes, genus, species, cultivars, varieties, variants and other minor taxonomic groups that lack a consistent nomenclature.

Expression (including transcription and translation) of BR-BioSig or particular fragments thereof may be regulated with respect to the level of expression, the tissue type(s) where expression takes place and/or developmental stage of expression. A number of heterologous regulatory sequences (e.g., promoters and enhancers) are available for controlling the expression of a BR-BioSig nucleic acid. These include constitutive, inducible and regulatable promoters, as well as promoters and enhancers that control expression in a tissue- or temporal-specific manner. Novel regulatory sequences containing known regulatory motifs and elements, or functional portions or fragments of known regulatory sequences could also be used. Exemplary constitutive promoters include the raspberry E4 promoter (U.S. Pat. Nos. 5,783,393 and 5,783,394), the 35S CaMV (Jones J D et al, (1992) Transgenic ResI:285-297), the CsVMV promoter (Verdaguer Bet al., PlantMol Biol (1998) 37:1055-1067) and the melon actin promoter (published PCT application W00056863). Exemplary tissue-specific promoters include the tomato E4 and E8 promoters (U.S. Pat. No. 5,859,330) and the tomato 2 AII gene promoter (Van Haaren M J J et al., Plant Mol Bio (1993) 21:625-640).

To produce transgenic plants of this invention, a construct comprising the gene encoding BR-BioSig, or nucleic acid encoding its functional equivalent, and a promoter are incorporated into a vector through methods known and used by those of skill in the art. The promoter can comprise all or part of SEQ ID NO:17. The construct can also include any other necessary regulators such as terminators or the like, operably linked to the coding sequence. It can also be beneficial to include a 5′ leader sequence, such as the untranslated leader from the coat protein mRNA of alfalfa mosaic virus (Jobling, S. A and Gehrke, L. (1987) Nature 325:622-625) or the maize chlorotic mottle virus (MCMV) leader (Lommel, S. A, et al. (1991) Virology 81:382-385). Those of skill in the art will recognize the applicability of other leader sequences for various purposes. Targeting sequences are also useful and can be incorporated into the constructs of this invention. A targeting sequence is usually translated into a peptide which directs the polypeptide product of the coding nucleic acid sequence to a desired location within the cell, such as to the plastid, and becomes separated from the peptide after transit of the peptide is complete or concurrently with transit. Examples of targeting sequences useful in this invention include, but are not limited to, the yeast mitochondrial presequence (Schmitz, et al. (1989) Plant Cell 1:783-791), the targeting sequence from the pathogenesis-related gene (PR-1) of tobacco (Comellisen, et al. (1986) EMBO J. 5:37-40), vacuole targeting signals (Chrispeels, M. J. and Raikhel, N. V. (1992) Cell 68:613-616), secretory pathway sequences such as those of the ER or Golgi (Chrispeels, M. J. (1991) Ann. Rev. Plant Physiol. Plant Mol. Biol. 42:21-53). Intraorganellar sequences may also be useful for internal sites, e.g., thylakoids in chloroplasts. Theg, S. M. and Scott, S. V. (1993) Trends in Cell Bioi. 3:186-190.

In addition to 5′ leader sequences, terminator sequences are usually incorporated into the construct. In plant constructs, a 3′ untranslated region (3′ UTR) is generally part of the expression plasmid and contains a polyA termination sequence. The termination region which is employed will generally be one of convenience, since termination regions appear to be relatively interchangeable. The octopine synthase and nopaline synthase termination regions, derived from the Ti-plasmid of A. tumefaciens, are suitable for such use in the constructs of this invention.

The transcriptional initiation region may provide for constitutive expression or regulated expression. In addition to the RD29A promoter, many promoters are available which are functional in plants. Constitutive promoters for plant gene expression include, but are not limited to, the octopine synthase, nopaline synthase, or marmopine synthase promoters from Agrobacterium, the cauliflower mosaic virus (35S) promoter, the figwort mosaic virus (FMV) promoter, and the tobacco mosaic virus (TMV) promoter. Constitutive gene expression in plants can also be provided by the glutamine synthase promoter (Edwards, et al. (1990) PNAS 87:3459-3463), the maize sucrose synthetase 1 promoter (yang, et al. (1990) PNAS 87:4144-4148), the promoter from the Rol-C gene of the TLDNA of Ri plasmid (Sagaya, et al. (1989) Plant Cell Physiol. 30:649-654), and the phloem-specific region of the pRVC-S-3A promoter (Aoyagi, et al. (1988) Mol. Gen. Genet. 213:179-185).

Heat-shock promoters, the ribulose-1,6-bisphosphate (RUBP) carboxylase small subunit (ssu) promoter, tissue specific promoters, and the like can be used for regulated expression of plant genes. Developmentally-regulated, stress-induced, wound-induced or pathogen-induced promoters are also useful. The regulatory region may be responsive to a physical stimulus, such as light, as with the RUBP carboxylase ssu promoter, differentiation signals, or metabolites. The time and level of expression of the sense or antisense orientation can have a definite effect on the phenotype produced. Therefore, the promoters chosen, coupled with the orientation of the exogenous DNA, and site of integration of a vector in the genome, will determine the effect of the introduced gene. As used herein, the term “regulatory region” or “promoter” refer to a sequence of DNA, commonly but not always upstream (5′) to the coding sequence of a structural gene, which controls the expression of the coding region by providing recognition and binding sites for RNA polymerase and/or other factors required for transcription to start at the correct site.

Specific examples of regulated promoters also include, but are not limited to, the low temperature Kinl and cor6.6 promoters (Wang, et al. (1995) Plant Mol. Bioi. 28:605; Wang, et al. (1995) Plant Mol. Bioi. 28:619-634), the ABA inducible promoter (Marcotte Jr., et al. (1989) Plant Cell 1:969-976), heat shock promoters, such as the inducible hsp70 heat shock promoter of Drosphilia melanogaster (Freeling, M., et al. (1985) Ann. Rev. of Genetics 19: 297-323), the cold inducible promoter from B. napus (White, T. C, et al. (1994) Plant Physiol. 106:917), the alcohol dehydrogenase promoter which is induced by ethanol (Nagao, R. T., et al., Miflin, B. J., Ed. Oxford Surveys of Plant Molecular and Cell Biology, Vol. 3, p 384-438, Oxford University Press, Oxford 1986), the phloem-specific sucrose synthase ASUSI promoter from Arabidopsis (Martin, et al. (1993) Plant J. 4:367-377), the ACSI promoter (RodriguesPousada, et al. (1993) Plant Cell 5:897-911), the 22 kDa zein protein promoter from maize (Unger, et al. (1993) Plant Cell 5:831-841), the psI lectin promoter of pea (de Pater, et al. (1993) Plant Cell 5:877-886), the phas promoter from Phaseolus vulgaris (Frisch, et al. (1995) Plant J. 7:503-512), the lea promoter (Thomas, T. L. (1993) Plant Cell 5:1401-1410), the E8 gene promoter from tomato (Cordes, et al. (1989) Plant Ce111:1025-1034), the PCNA promoter (Kosugi, et al. (1995) Plant J. 7:877-886), the NTP303 promoter (Weterings, et al. (1995) Plant J. 8:55-63), the OSEM promoter (Hattori, et al. (1995) Plant J. 7:913-925), the ADP GP promoter from potato (Muller-Rober, et al. (1994) Plant Cell 6:601-604), the Myb promoter from barley (Wissenbach, et al. (1993) Plant J. 4:411-422), and the plastocyanin promoter from Arabidopsis (Vorst, et al. (1993) Plant J. 4:933-945).

Organ-specific promoters are also well known. For example, the patatin class I promoter is transcriptionally activated only in the potato tuber and can be used to target gene expression in the tuber (Bevan, M., 1986, Nucleic Acids Research 14:4625-4636). Another potato-specific promoter is the granule-bound starch synthase (GBSS) promoter (Visser, R. G. R, et al., 1991, Plant Molecular Biology 17:691-699). Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, P, 1986, Trans. R. Soc. London B314:343).

In another preferred embodiment, inhibition of endogenous BR-BioSig function is under control of regulatory sequences from genes whose expression is associated with drought stress. For example, when the promoter of the drought stress responsive Arabidopsis rd29A gene was used to drive expression of DREBIA, Arabidopsis plants were more tolerant to drought, salt and freezing stress and did not have the stunted stature associated with plants over-expressing the DREB1A gene from the CaMV 35S promoter (Kasuga et al, 1999 Nature Biotech 17: 287). Promoters from other Arabidopsis genes that are responsive to drought stress, such as COR47 (Welinet al. 1995, Plant Mol. Biol. 29: 391), KINI (Kurkela and Franck, 1990, Plant Mol. Biol. 15: 137), RD22BP (Abe et al. 1997, Plant Cell 9, 1859), ABA1 (Accession NumberAAGI7703), and ABA3 (Xiong et al. 2001, Plant Cell 13:2063), could be used. Promoters from drought stress inducible genes in other species could be used also. Examples are the rab17, ZmFer1 and ZmFer2 genes from maize (Bush et al., 1997 Plant J 11:1285; Fobis-Loisy, 1995 Eur J Biochem 231:609), the tdi-65 gene from tomato (Harrak, 2001 Genome 44:368), the His1 gene of tobacco (Wei and O'Connell, 1996 Plant Mol Biol 30:255), the Vupat1 gene from cowpea (Matos, 2001 FEBS Lett 491:188), and CDSP34 from Solanum tuberosum (Gillet et al, 1998 PlantJ 16:257).

Exemplary methods for inhibiting the expression of endogenous BR-BioSig in a host cell include, but are not limited to antisense suppression (Smith, et al., Nature (1988) 334:724-726; van der Krol et al., Biotechniques (1988) 6:958-976); co-suppression (Napoli, et al, Plant Cell (1990) 2:279-289); ribozymes (PCT Publication WO 97/1032S); and combinations of sense and antisense (Waterhouse, et al., Proc. Natl. Acad. Sci. USA (1998) 95:13959-13964). Methods for the suppression of endogenous sequences in a host cell typically employ the transcription or transcription and translation of at least a portion of the sequence to be suppressed. Such sequences may be homologous to coding as well as non coding regions of the endogenous sequence. Antisense inhibition may use the entire cDNA sequence (Sheehy et al., Proc. Natl. Acad. Sci. USA (1988) 85:8805-8809), a partial cDNA sequence including fragments of 5′ coding sequence, (Cannon et al., Plant Molec. Biol. (1990) 15:39-47), or 3′ non-coding sequences (Ch'ng et al., Proc. Natl. Acad. Sci. USA (1989) 86:10006-1001 0). Co-suppression techniques may use the entire cDNA sequence (Napoli et al., supra; vander Krol et al., The Plant Cell (1990) 2:291 299), or a partial cDNA sequence (Smith et al., Mol. Gen. Genetics (1990) 224:477-481).

In addition to the antisense nucleic acids of the present invention, oligonucleotides can be constructed which will bind to duplex nucleic acid either in the gene or the DNA:RNA complex of transcription, to form a stable triple helix containing or triplex nucleic acid to inhibit transcription and/or expression of a gene encoding an BR-BioSig polypeptide or its functional equivalent (Frank-Kamenetskii, M. D. and Mirkin, S. M. (1995) Ann. Rev. Biochem. 64:65-95). Such oligonucleotides can be constructed using the base-pairing rules of triple helix formation and the nucleotide sequence of the gene or mRNA for Ftase. These oligonucleotides can block BR-BioSig-type activity in a number of ways, including prevention of transcription of the gene or by binding to mRNA as it is transcribed by the gene.

A particular aspect of the invention pertains to the use of post transcriptional gene silencing (PTGS) to repress gene expression. Double stranded RNA can initiate the sequence specific repression of gene expression in plants and animals. Double stranded RNA is processed to short duplex oligomers of 21-23 nucleotides in length. These small interfering RNA's suppress the expression of endogenous and heterologous genes in a sequence specific manner (Fire et al. Nature 391:806-811, Carthew, Curr. Opin. in Cell Biol., 13:244-248, Elbashir et al., Nature 411:494-498). An RNAi suppressing construct can be designed in a number of ways, for example, transcription of an inverted repeat which can form a long hair pin molecule, inverted repeats separated by a spacer sequence that could be an unrelated sequence such as GUS or an intron sequence. Transcription of sense and antisense strands by opposing promoters or co-transcription of sense and antisense genes is also possible and encompassed in the scope of the present invention.

Standard molecular and genetic tests may be performed to further analyze the association between a gene and an observed phenotype. Exemplary techniques are described below.

### 1. DNA/RNA Analysis

The stage- and tissue-specific gene expression patterns in mutant versus wild-type lines may be determined, for instance, by in situ hybridization. Analysis of the methylation status of the gene, especially flanking regulatory regions, may be performed. Other suitable techniques include overexpression, ectopic expression, expression in other plant species and gene knockout (reverse genetics, targeted knock-out, viral induced gene silencing [VIGS, see Baulcombe D, Arch Virol Suppl (1999) 15:189-201]).

In a preferred application expression profiling, generally by microarray analysis, is used to simultaneously measure differences or induced changes in the expression of many different genes. Techniques for micro array analysis are well known in the art (Schena M et al., Science (1995) 270:467-470; Baldwin D et al., (1999) Cur Opin Plant Bioi. 2(2):96-103; Dangond F, Physiol Genomics (2000) 2:53-58; van Hal N L et al., J Biotechnol (2000) 78:271-280; Richmond T. and Somerville S., Cuff Opin Plant Biol (2000) 3:108-116). Expression profiling of individual tagged lines may be performed. Such analysis can identify other genes that are coordinately regulated as a consequence of the overexpression of the gene of interest, which may help to place an unknown gene in a particular pathway.

### 2. Gene Product Analysis

Analysis of gene products may include recombinant protein expression, antisera production, immunolocalization, biochemical assays for catalytic or other activity, analysis of phosphorylation status, and analysis of interaction with other proteins via yeast two-hybrid assays.

### 3. Pathway Analysis

Pathway analysis may include placing a gene or gene product within a particular biochemical, metabolic or signaling pathway based on its mis-expression phenotype or by sequence homology with related genes. Alternatively, analysis may comprise genetic crosses with wild-type lines and other mutant lines (creating double mutants) to order the gene in a pathway, or determining the effect of a mutation on expression of downstream “reporter” genes in a pathway.

Further Methods for Generation of Mutated Plants with a Drought-Resistant Phenotype

The invention further provides a method of identifying plants that have mutations in endogenous BR-BioSig that confer increased drought-resistance, and generating drought-resistant progeny of these plants that are not genetically modified. In one method, called “TILLING” (for targeting induced local lesions in genomes), mutations are induced in the seed of a plant of interest, for example, using EMS treatment. The resulting plants are grown and self-fertilized, and the progeny are used to prepare DNA samples. BR-BioSig specific PCR is used to identify whether a mutated plant has a BR-BioSig mutation. Plants having BR-BioSig mutations may then be tested for drought-resistance, or alternatively, plants may be tested for drought-resistance, and then BR-BioSig-specific PCR is used to determine whether a plant having increased drought-resistance has a mutated BR-BioSig gene. TILLING can identify mutations that may alter the expression of specific genes or the activity of proteins encoded by these genes (see Colbert et al (2001) Plant Physiol 126:480-484; McCallumet al (2000) Nature Biotechnology 18:455-457).

In another method, a candidate gene/Quantitative Trait Locus (QTLs) approach can be used in a marker assisted breeding program to identify alleles of or mutations in the BR-BioSig gene or orthologs of BR-BioSig that may confer increased resistance to drought (see Foolad et al., Theor Appl Genet. (2002) 104(6-7):945-958; Rothan et al., Theor Appl Genet (2002) 105(1):145-159); Dekkers and Hospital, NatRev Genet. (2002) January; 3(1):22-32). Thus, in a further aspect of the invention, a BR-BioSig nucleic acid is used to identify whether a drought-resistant plant has a mutation in endogenous BR-BioSig.

### Examples

### Isolation of Cyp85a2 Knockout Mutant

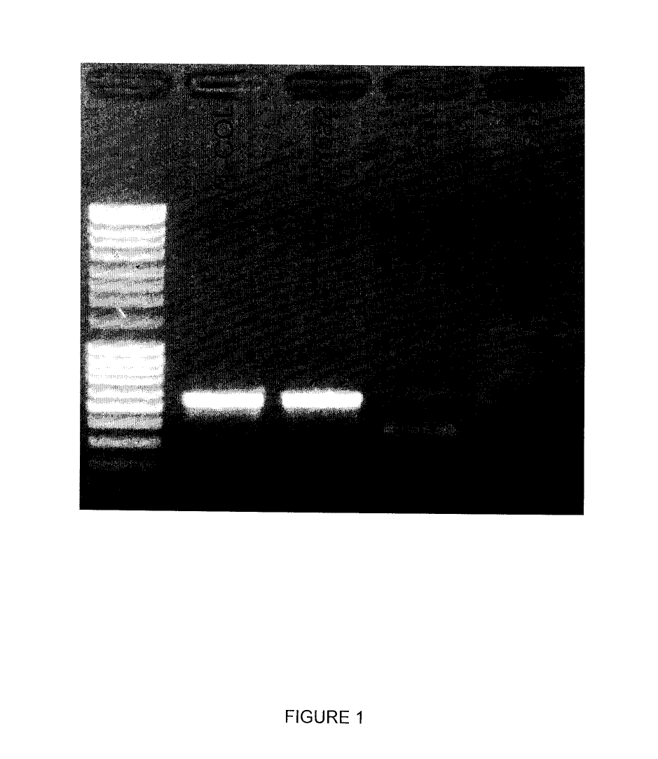

The homozygous T-DNA insertional mutant cyp85a2 (SALK_{—}129352) was discovered on the Salk SIGnAL Web site (http://signal.salk.edu) and obtained from the ABRC (Columbus, Ohio). Although it has been previously published that SALK_{—}129352 is a KO in CYP85A2, it was necessary to confirm this by checking for CYP85A2 expression in young seedlings where BR production is high. RT-PCR analysis using RNA isolated from the cyp85a2 mutant and wild-type plants showed that indeed there is a lack of expression of CYP85A2 in cyp85a2 indicative of a null allele (FIG. 1).

### Drought Resistance in Cyp85a2 Knockout Mutant:

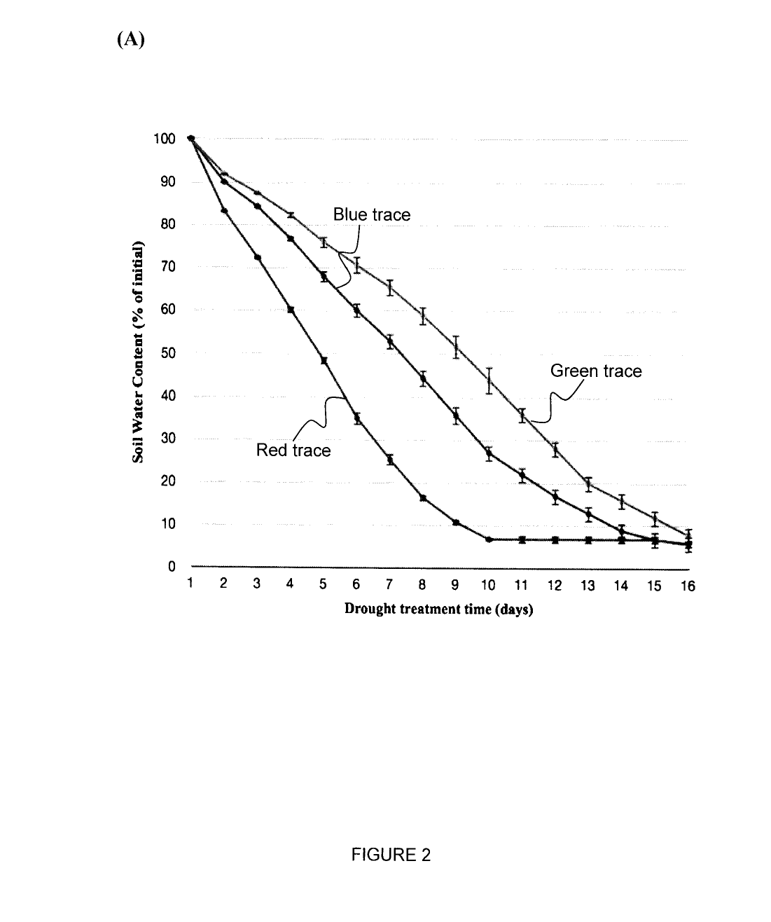

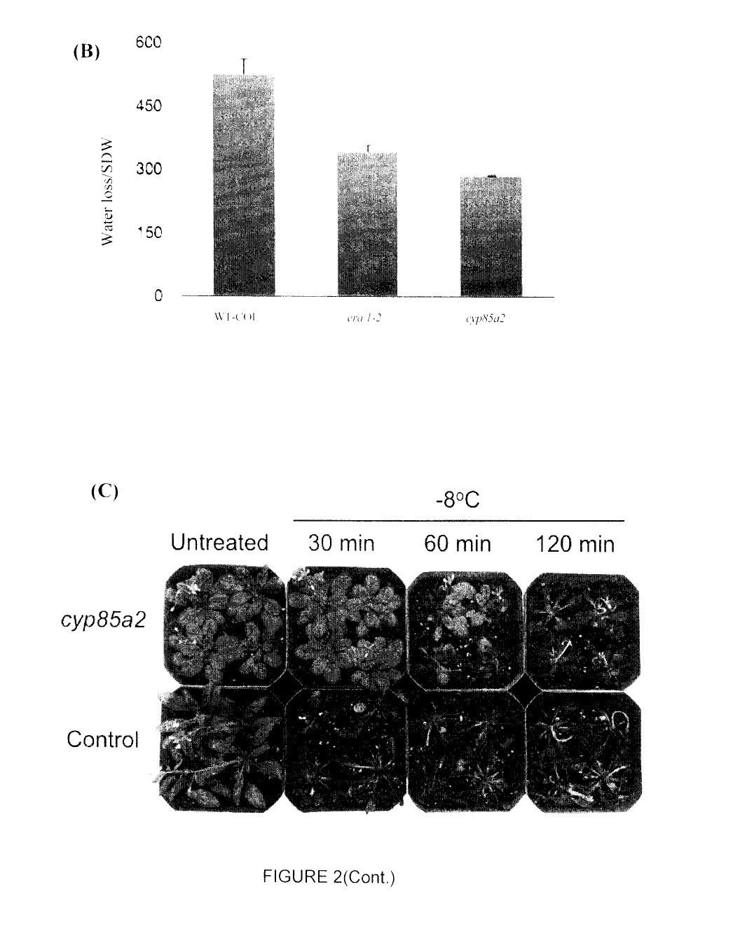

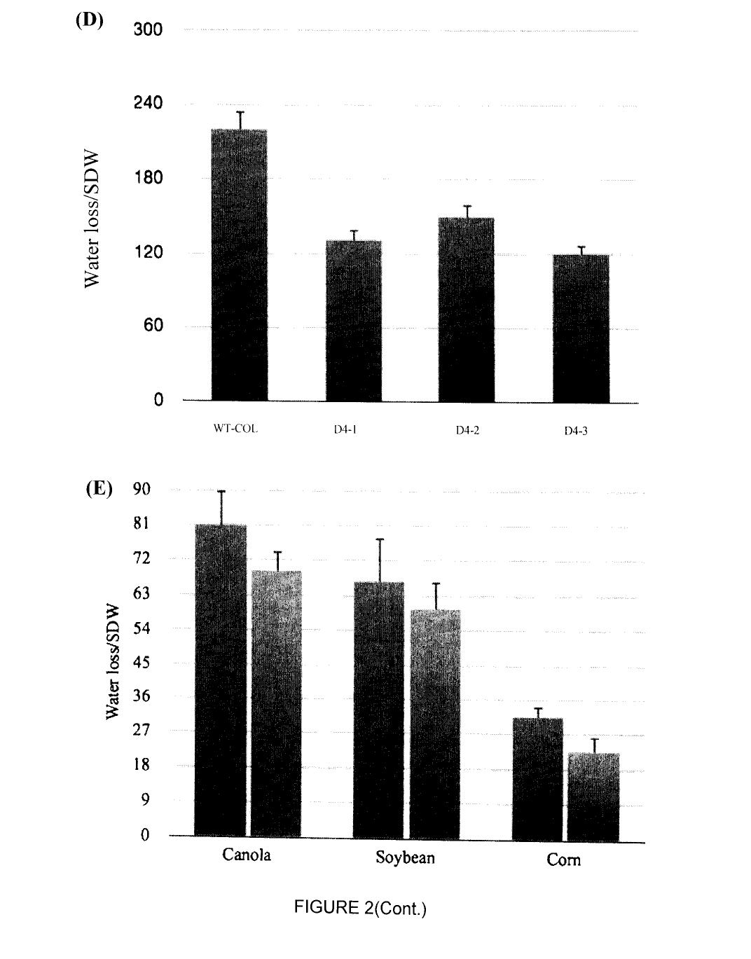

Soil (20:20:20), prepared that day, was consistently weighed into pots (typically 140 g) and covered with a plastic wrap (GLAD Press'n Seal® Wrap) that had been punctured with a small hole in the centre. Seeds were stratified on moist filter paper for 4 days and allowed to germinate for approximately 2 days before individual germinating seeds were placed on soil. Plants were grown under optimal conditions (22 C (71.6 F), 16 hr light of 200uE, 60% RH) and were watered daily until the first open flower was observed. Before drought treatment, pots were watered to a set weight and drought treatment was started on day 0 by withholding water for 16 days (FIG. 2A). The homozygous mutant cyp85a2 exhibited higher soil water content than the wild-type control, but lower soil water content than the mutant era1-2 during induced drought, suggesting that this line loses less water than the control under these conditions. However, these measurements fail to account for subtle differences in plant size. To get a more accurate sense of a plant's drought stress resistance and water use efficiency, it is therefore necessary to normalize the data based on the size of the plant (e.g. final shoot dry weight of the plant). This was typically done by withholding water for 9 days after which shoot biomass was harvested and fresh weight was determined. Shoots were then dried at 60 C for 3 days and dry weight was determined. The ratio of decrease in soil water content after a period of drought to the final shoot dry weight is an accurate normalized calculation of total water loss. It is important to note that this normalized value of the drought response is only relevant for a particular experiment since either total water loss, or final shoot dry weight can vary significantly over various experimental conditions (i.e. from experiment to experiment).

The Arabidopsis mutant era1-2 is extremely drought resistant, and is described in U.S. Pat. No. 7,262,338B2. The subsequent incorporation of this technology into several crops has proven to be very useful for improving yield under drought conditions, making it an industry standard and benchmark in comparative testing for drought resistance.

By using the aforementioned normalized calculation, the cyp85a2 line demonstrates significantly less (P<0.05) water loss per unit of shoot weight than that of era1-2 (FIG. 2B). This result supports the interpretation that line cyp85a2 is more drought resistant and has higher water use efficiency than wild-type columbia and era1-2. The cyp85a2 was tested as a representative gene of the cyp85 gene family involved in BR biosynthesis, and it shares significant sequence identity and similarity to the cyp85a1 gene. They share 83% identity and 92% similarity.

### Cold Resistance in Cyp85a2 Knockout Mutant:

Plants were grown in 3 inch pots under optimal conditions (22 C (71.6 F), 18 hr light of 200 uE, 60% RH) in a growth chamber until appearance of the first flower. A cold stress treatment was applied at −8° C. (17.6 F) for 30, 60, and 120 minutes following an overnight acclimation at 4° C. (39.2 F) (FIG. 2C). The cyp85a2 mutant was clearly able to survive the −8° C. (17.6 F) treatment after 30 minutes, remaining green and turgid, whereas the wild-type columbia control plants had already wilted.

### Heat Resistance in Cyp85a2 Knockout Mutant:

Plants were grown in 3 inch pots under optimal conditions (22 C (71.6 F), 24 hr light of 200 uE, 60% RH) in a growth chamber until appearance of the first flower. A heat stress treatment was applied by placing plants at 42 C (107.6 F) for 2 hours. One week following the stress period the plants were assessed for number of aborted flowers. 100 siliques were assessed and there was a 6% decrease in silique abortion for cyp85a2 relative to wild-type columbia (WT-COL).

Construction and Generation of the CYP90a1/DWF4 RNAi Transgenic Lines:

Standard methods were used for the cloning and generation of Arabidopsis RNAi transgenic plants. Briefly, the CYP90B1/DWF4 RNAi construct was cloned into modified pCAMBIA vector (named p1667) which contains a stress-inducible promoter, RD29A, and a NOS terminator flanking the gene of interest. RD29A promoter drives the expression of the engineered constructs under the induced condition.

Cloning of the above-mentioned fragment was carried out using In-Fusion HD Cloning kit [Clontech Laboratories; Cat #639648], following manufacturer's instructions. Two-step recombination cloning was performed by inserting sense and antisense orientation of the gene fragments sequentially, each of the sense strands containing the ‘intron spacer’ sequence at the end. Briefly, an In-Fusion reaction is set up with linearized vector and PCR amplified inserts. The primers designed for amplifying the inserts were gene-specific primers with a 15 bp extension complementary to the vector ends.

The sense and antisense inserts were prepared with PCR amplification using Phusion Hot Start II High-Fidelity DNA Polymerase [Finnzymes; Cat# F-549S] using the following four primers, P1 to P4 listed in the 5′ to 3′ direction.

(SEQ ID NO: 18)
P1; TGTCTAGAGGATCCCCCTTTTGGGATGGGTT
(SEQ ID NO: 19)
P2; TTCGAGCTCGGTACCCGGGGCGAATTCCTATGAGCTG
(SEQ ID NO: 20)
P3; CATAGGAATTCGCCCCGGGTCCCCACGTCGAAAA
(SEQ ID NO: 21)
P4; TTCGAGCTCGGTACCCGGGCTTTTGGGATGGGTT

On the other hand, the linearized vector was prepared by digesting p1667 with SmaI. Both the vector and inserts were gel-purified. Primer P1 is the forward primer for the sense strand amplification. It contains 16 bp of the gene sequence with 15 bp of the flanking RD29A promoter sequence. P2 is designed as a common reverse primer for each of the sense strands. It contains 19 bp of vector-specific sequence and 18 bp of an intron sequence that is specific to the sense strands. P3 and P4 are the forward and reverse primers, respectively, for amplifying the antisense strands. P3 used for amplifying the antisense orientation of the gene has 18 bp extension complementary to the intron sequence. P4 has identical gene-specific sequence as of P1 and the vector-specific sequence similar to that in P2.

Subsequently, the sense insert was cloned into the linearized vector, p1667, keeping the SmaI restriction site undisrupted. The reaction was prepared as follows:

| | | |
|---|---|---|
| 5X In-Fusion HD Enzyme Premix | 2 | μl |
| Linearized Vector | 400 | ng |
| Purified PCR Fragment | 50-60 | ng |
| dH2O (as required) | x | μl |
| Total Volume | 10 | μl |

The reaction was incubated at 50° C. for 15 min.

Bacterial transformation went as follows: 5 μl of the reaction was transformed into 50 μl of Stellar competent cells (Clontech Laboratories; Cat#639763) following the manufacturer's instructions and selected on LB plates containing Kanamycin (50 μg/ml).

The positive clones were confirmed by colony PCR using a combination of insert and vector-specific primers as follows:

(SEQ ID NO: 22)
RD29A FW: 5′-GTGAGACCCTCCTCTGTTTTAC-3′
(SEQ ID NO: 19)
P2 5′-TTCGAGCTCGGTACCCGGGGCGAATTCCTATGAGCTG-3′

Plasmid DNA was isolated from the overnight cultures of the positive clones, digested with SmaI and the antisense insert cloned following the same method described above.

Primers used for selecting the positive clones for insertion of antisense strands were:

(SEQ ID NO: 20)
P3 5′-CATAGGAATTCGCCCCGGGTCCCCACGTCGAAAA-3′
(SEQ ID NO: 23)
SLR RV: 5′-CGCAAGACCGGCAACAGGATT-3′

SLR RV Primer is specific to NOS terminator.

Mobilizing the RNAi constructs into Agrobacterium tumefaciens went as follows: Plasmid DNA of the binary vector p1667 containing the RNAi constructs were isolated and mobilized into Agrobacterium tumefaciens GV3101 strain following standard freeze-thaw method and selected on LB plates containing Kanamycin (50 μg/ml) and Rifampicin (50 μg/ml). The positive colonies were confirmed by colony PCR with the above-mentioned primers.

Plant transformation went according to standard protocols: The A. tumefaciens harboring the respective RNAi constructs were grown in LB broth containing Kanamycin (50 μg/ml) and Rifampicin (50 μg/ml) for 2 days. This was used for genetic transformation of Arabidopsis thaliana ecotype Columbia-0 by standard floral dip method. Briefly, the bacterial pellet was resuspended in ½MS medium with 5% sucrose, 0.2% Silwet L-77 was added and the flowers of 3-weeks-old A. thaliana was dipped into the prepared culture twice with a five-day interval.

### Drought Resistance in the CYP90B1/DWF4 RNAi Mutants

Three independent transgenic RNAi lines (D4-1, D4-2, and D4-3) specifically targeting the CYP90B1/DWF4 gene were also tested for drought resistance (FIG. 2D). Again the ratio of decrease in soil water content after a period of drought to the final shoot dry weight was used as a normalized calculation of total water loss, and therefore an accurate measure of drought resistance. All three RNAi lines targeting the CYP90B1/DWF4 gene showed improved drought resistance and had higher water use efficiency than wild-type. The CYP90B1/DWF4 was tested as a representative gene of the CYP90 gene family (i.e. CYP90A1/CPD, CYP90C1/ROT3, CYP90D1) since they all are involved in the BR biosynthesis pathway and share homology to one another.

### Lack of BRs, Through Chemical Inhibition of DWF4, Improve the Drought Resistance in Corn, Soybean, and Canola

The observation that a deficiency in endogenous BL in cyp85a2 enables drought resistance to Arabidopsis prompted the hypothesis that a similar mechanism might exist in other plant species, such as corn, soybean, and canola. To test whether the lack of endogenous BRs could improve the drought response of corn (B73 cultivar), soybean (OAC Wallace cultivar), and canola (Westar cultivar) under drought treatment, chemical inhibition of the BR biosynthetic pathway by BRZ was used to inhibit BR production. Brassinazole (BRZ) specifically blocks BL biosynthesis by inhibiting the cytochrome P450 steroid C-22 hydroxylase encoded by the DWF4/CYP90B1 gene (Asami et al., 2001). For these experiments a stock solution of BRZ was made at 40 μM and allowed to soak into the soil for a final concentration of 10 μM BRZ for canola and soybean and 20 μM BRZ for corn. As for the previous drought experiments in Arabidopsis where all plants were grown to the start of flowering, and then subjected to a drought stress treatment, one plant per pot, where water was withheld, several modifications to the experiment are worth noting. All pots had an initial start weight of 260 g. Canola and soybean were grown for 6.5 and 5 weeks, respectively, and flowered during the course of the 4 day experiment. Alternatively, corn was grown for five weeks but did not flower during the 3.5 day experiment. Immediately preceding the start of the experiment, daily measurements of water use were monitored to quantify the rate of water loss. To prolong the experiment from 2 to 4 days, in the case of canola and soybean, 50 mL of a 10 μM BRZ solution was added to the pots for the first 3 days. Indeed, for each plant type, chemical treatment (through root uptake) resulted in significantly less (P<0.05) water loss per unit of shoot weight than that of the untreated controls (FIG. 2E). Specifically, the water use efficiency improved by 28% for corn (20 μM final concentration of chemical), 14% for soybean (10 μM final), and 15% for canola (10 μM final). These results demonstrate that the application of BRZ is an effective means of improving the water use efficiency of corn, soybean, and canola under water limiting conditions.

### Related Abiotic and Osmotic Tolerance/Resistance Mechanisms

During a typical life cycle, plants are often exposed to unfavorable environmental conditions that may interrupt or disturb the normal growth, development, or productivity they accomplish under optimal growth conditions. Environmental stresses can be either biotic or abiotic. Abiotic stresses of particular interest include drought, high salinity, and extremes in temperature. Interestingly, a common component of drought, high salinity, and low temperature stress is water deficit, which occurs when the rate of transpiration exceeds water uptake (Bray, 1993). Water-deficit stress can be defined as a situation in which the optimal physiological functioning of the plant is compromised from a reduction in water potential and turgor. Severe changes of water potential in the plant environment can then cause an osmotic stress, disturbing the normal cellular functioning, and eventually leading to cell death. Drought conditions cause water deficits simply by reducing the amount of available water for plant growth. Under conditions of high salinity, where water may not be limiting, the presence of high salt concentrations can make it more difficult to extract water from the surrounding environment. Extremely low temperatures that result in freezing also lead to water deficit through cellular dehydration caused by water leaving the cells to form ice crystals in intercellular spaces. At the cellular level, water deficit can cause changes in cell volume and membrane shape, disruption of membrane integrity, disruption of water potential gradients, loss of turgor, altered concentrations of solutes, and the denaturation of proteins. The plant responds by regulating its homeostasis through a number of physiological, cellular and biochemical changes, including changes in cell wall architecture, membrane structure and function, tissue water content, gene and protein expression, lipids, and primary and secondary metabolite composition (reviewed in Bartels and Sunkar, 2005). More specifically, drought triggers alterations in root and shoot development, photosynthetic capacity, ion transport, gene expression, the accumulation of metabolites such as ABA and osmotically active compounds, and the accumulation of protective proteins (Ramachandra Reddy et al., 2004; Xiong et al., 2002).

Gene expression profiling, or transcriptomics, using cDNA microarrays or gene chips has identified hundreds of genes that are regulated by abiotic stress (Shinozaki et al. 2003; Seki et al. 2004). Analysis of this expression data has contributed greatly to our understanding of the genes and regulatory networks that contribute to these inter-related environmental stresses. In one particular microarray study, using approximately 7000 independent Arabidopsis full-length cDNAs, the authors identified 299 drought-inducible genes, 54 cold-inducible genes, 213 high salinity inducible genes and 245 ABA-inducible genes (ABA is hormone induced by stress) (Seki et al., 2002a, Seki et al., 2002b). More than half of the drought-inducible genes are also induced by high salinity and/or ABA treatments, indicating the existence of significant crosstalk among the drought, high-salinity and ABA responses. Fewer drought-inducible genes were also induced by cold stress. These results supported previous models demonstrating the overlap of gene expression in response to drought, high salinity, cold and ABA. Many transcription-factor genes were found among the stress-inducible genes, suggesting that various transcriptional regulatory mechanisms function in the drought, cold or high salinity stress signal transduction pathways (Seki et al., 2002a, Seki et al., 2002b, Chen et al., 2002, Fowler et al., 2002, Krebs et al., 2002). These stress-inducible transcription factors include members of the DRE-binding protein (DREB/CBF) family, the ethylene-responsive element binding factor (ERF) family, the zinc-finger family, the WRKY family, the MYB family, the basic helix-loop-helix (bHLH) family, the basic-domain leucine zipper (bZIP) family, the NAC family, and the homeodomain transcription factor family. These transcription factors could regulate various stress-inducible genes cooperatively or separately, and may constitute gene networks involved in responses to drought, cold and high salinity stresses. Interestingly, over-expression of the CBF/DREB transcription factors confers improved freezing, drought, and salt tolerance, further highlighting the extensive cross-talk and inter-relatedness of these various abiotic stresses (Jaglo-Ottosen et al. 1998; Liu et al. 1998; Kasuga et al. 1999; Shinozaki and Yamaguchi-Shinozaki 2000; Jaglo et al. 2001; Thomashow 2001).

The capability of plants to survive and recuperate from an abiotic stress is a function of basal and acquired tolerance mechanisms. The process of acquiring tolerance to a given stress condition is known as acclimation, whereby following an exposure to moderate stress conditions the overall stress tolerance of the plant is transiently improved upon (Hallberg et al. 1985; Guy 1999; Thomashow 1999). For example, if plants are pre-exposed to a non-lethal low temperature, they can acquire enhanced tolerance to otherwise lethal low temperatures, known as acquired freezing tolerance. Likewise, enhanced tolerance to heat stress can be achieved if a plant is pre-exposed to non-lethal high temperature, known as acquired thermotolerance. During temperature acclimation, a plant alters its homeostasis through a number of physiological, cellular and biochemical changes, including changes in cell wall architecture, membrane structure and function, tissue water content, gene and protein expression, lipids, and primary and secondary metabolite composition (Gilmour et al. 2000; Shinozaki and Dennis 2003). Although it is convenient to treat high and low temperature as separate stress factors, they are in fact interrelated, and share a common set of cellular, biochemical and molecular responses, such that plants can display cross-tolerance. For example, it was observed many years ago that some cold tolerant plants were also more thermotolerant (Levitt, 1972). In addition, it has also been observed that application of a heat shock seems to improve chilling tolerance in a number of cold-sensitive species (Saltveit, 2002; Saltveit and Hepler, 2004; Saltveit et al., 2004) In general, tolerance to one stress is induced by acclimation to the other. Plants therefore possess stress-specific adaptive responses as well as responses which are protective for more than one environment stress (Chinnusamy et al., 2004). Once again, this is not so surprising given the fact that abiotic stresses, such as drought, salinity, cold, freezing, and high temperature eventually lead to an osmotic stress (i.e. severe changes in water potential) within the plant.

While the invention has been described with reference to specific methods and embodiments, it will be appreciated that various modifications and changes may be made without departing from the invention. All publications cited herein are expressly incorporated herein by reference for the purpose of describing and disclosing compositions and methodologies that might be used in connection with the invention. All cited patents, patent applications, and sequence information in referenced web sites and public databases are also incorporated by reference.

### REFERENCES

Guy, C. (1999). “Molecular responses of plants to cold shock and cold acclimation.” J Mol Microbiol Biotechnol 1(2): 231-42.
Hallberg, R. L., Kraus, K. W. and Hallberg, E. M. (1985). “Induction of acquired thermotolerance in Tetrahymena thermophila: effects of protein synthesis inhibitors.” Mol Cell Biol 5(8): 2061-9.
Thomashow, M. F. (1999). “PLANT COLD ACCLIMATION: Freezing Tolerance Genes and Regulatory Mechanisms.” Annu Rev Plant Physiol Plant Mol Biol 50: 571-599.
Shinozaki, K. and Dennis, E. S. (2003). “Cell signalling and gene regulation: global analyses of signal transduction and gene expression profiles.” Curr Opin Plant Biol 6(5): 405-9.
Gilmour, S. J., Sebolt, A. M., Salazar, M. P., Everard, J. D. and Thomashow, M. F. (2000). “Overexpression of the Arabidopsis CBF3 transcriptional activator mimics multiple biochemical changes associated with cold acclimation.” Plant Physiol 124(4): 1854-65.
Levitt J (1972) Responses of plants to environmental stresses. Academic Press, New York.
Saltveit M E (2002) “Heat shocks increase the chilling tolerance of rice seedling radicles.” J. Agric Food Chem 50:3232-3235.
Saltveit M E, Helper P K (2004) “Effect of heat shock on the chilling sensitivity of trichomes and petioles of African violet” Physiol Plant 121:35-43.
Chinnusamy, V., Schumaker, K., and Zhu, J. K. (2004). Molecular genetic perspectives on cross-talk and specificity in abiotic stress signalling in plants. J Exp Bot 55, 225-236.
Seki M, Narusaka M, Ishida J, Nanjo T, Fujita M, Oono Y, Kamiya A, Nakajima M, Enju A, Sakurai T et al.: Monitoring the expression profiles of 7000 Arabidopsis genes under drought, cold, and high-salinity stresses using a full-length cDNA microarray. Plant J 2002, 31:279-292.
Seki M, Ishida J, Narusaka M, Fujita M, Nanjo T, Umezawa T, Kamiya A, Nakajima M, Enju A, Sakurai T et al.: Monitoring the expression pattern of ca. 7000 Arabidopsis genes under ABA treatments using a full-length cDNA microarray. Funct Integ Genom 2002, 2:282-291.
Chen W, Provart N J, Glazebrook J, Katagiri F, Chang H S, Eulgem T, Mauch F, Luan S, Zou G, Whitham S A et al.: Expression profile matrix of Arabidopsis transcription factor genes suggests their putative functions in response to environmental stresses. Plant Cell 2002, 14:559-574.
Fowler S, Thomashow M F: Arabidopsis transcriptome profiling indicates that multiple regulatory pathways are activated during cold acclimation in addition to the CBF cold response pathway. Plant Cell 2002, 14:1675-1690.
Krebs J A, Wu Y, Chang H S, Zhu T, Wang X, Harper J: Transcriptome changes for Arabidopsis in response to salt, osmotic, and cold stress. Plant Physiol 2002, 130:2129-2141.

1. A nucleic acid construct for inducing an increase in growth or abiotic stress tolerance in a plant under stress conditions, or a cell of said plant, comprising at least one promoter operably linked to at least one nucleic acid that ultimately inhibits the polynucleotide expression or polypeptide function of: a polynucleotide as defined in SEQ ID NO:1, SEQ ID NO:3, SEQ ID NO:5, SEQ ID NO:7 SEQ ID NO:9, SEQ ID NO:11, SEQ ID NO:13, or SEQ ID NO:15; a polynucleotide encoding a polypeptide as defined in SEQ ID NO:2, SEQ ID NO:4, SEQ ID NO:6 SEQ ID NO:8, SEQ ID NO:10, SEQ ID NO:12, SEQ ID NO:14, or SEQ ID NO:16; a polynucleotide having at least 40% sequence identity to a polynucleotide as defined in SEQ ID NO:1, SEQ ID NO:3, SEQ ID NO:5, SEQ ID NO:7 SEQ ID NO:9, SEQ ID NO:11, SEQ ID NO:13, or SEQ ID NO:15; and a polynucleotide encoding a polypeptide having at least 40% sequence identity to a polypeptide as defined in SEQ ID NO:2, SEQ ID NO:4, SEQ ID NO:6 SEQ ID NO:8, SEQ ID NO:10, SEQ ID NO:12, SEQ ID NO:14, or SEQ ID NO:16.

2-44. (canceled)
